(19) United States
(12) Patent Application Publication
(10) Pub. No.: US 2018/0056131 A1
(43) Pub. Date: Mar. 1, 2018

(54) EXERCISE SUPPORT SYSTEM, EXERCISE SUPPORT METHOD, EXERCISE SUPPORT PROGRAM, AND EXERCISE SUPPORT DEVICE

(71) Applicant: SEIKO EPSON CORPORATION, Tokyo (JP)

(72) Inventors: Hironori NAKAZAWA, Matsumoto-shi (JP); Masao KURODA, Shiojiri-shi (JP)

(73) Assignee: SEIKO EPSON CORPORATION, Tokyo (JP)

(21) Appl. No.: 15/684,246

(22) Filed: Aug. 23, 2017

(30) Foreign Application Priority Data
Aug. 30, 2016 (JP) 2016-167625

(51) Int. Cl. A63B 24/00; A63B 71/06; A61B 5/024; A61B 5/00

(52) U.S. Cl. CPC A63B 24/0075; A63B 71/0616; A63B 24/0062; A63B 71/0622; A61B 5/02438; A63B 2024/0078; A61B 5/681; A61B 5/7282; A63B 2230/062; A63B 2225/50; A63B 2071/0663; A61B 5/02405

(57) ABSTRACT

A mobile terminal device as an exercise support device includes: an event information acquisition unit which acquires event information about an event for a user; an activity menu acquisition unit which acquires practice day information leading up to the event and an exercise menu; an exercise plan generation unit which generates an exercise plan using the event information, the practice day information, and the exercise menu; a pulse wave information acquisition unit which acquires pulse wave information of the user; and a physical condition determination unit which determines physical condition of the user based on the pulse wave information. The exercise plan generation unit modifies the exercise menu or the exercise plan, based on a result of the determination by the physical condition determination unit.

### CROSS-REFERENCE TO RELATED APPLICATION

This application claims priority to JP 2016-167625 filed Aug. 30, 2016, which is expressly incorporated herein by reference in its entirety.

### BACKGROUND

### 1. Technical Field

The present invention relates to an exercise support system, an exercise support method, an exercise support program, and an exercise support device.

### 2. Related Art

According to the related art, a technique of calculating depths of sleep based on heart rate and respiration rate and calculating the amount of recovery of the user's body by multiplying deep sleep equivalent to sleep stage 4, of the calculated depths of sleep, by the ratio of REM sleep or the cumulative time of REM sleep, is disclosed (see, for example, WO2013/161072). Using such a technique, the current recovery state of the user can be estimated.

However, with the foregoing technique, the user must carry out training while checking the recovery state by him/herself. Therefore, the user may overtrain or suffer injuries and may not be able to achieve sufficient training effects.

### SUMMARY

An advantage of some aspects of the invention is to solve at least a part of the problems described above, and the invention can be implemented as the following configurations or application examples.

### APPLICATION EXAMPLE 1

An exercise support device according to this application example includes: a memory storing a program; and a processor. When performing the program, the processor functions as: an event information acquisition unit which acquires event information about an event in which a user plans to participate; an activity menu acquisition unit which acquires practice day information identifying the practice day on which the user will exercise before the event, and exercise information representing a value of a parameter of the exercise to be performed on the practice day; a pulse information acquisition unit which acquires pulse information generated by a pulse sensor about a pulse of the user; a physical condition determination unit which determines a physical condition of the user based on the pulse information; and an exercise plan generation unit which generates an exercise plan identifying the practice day and the value of the parameter of the exercise to be performed on the practice day, using a result of the determination on the physical condition of the user by the physical condition determination unit, the event information, the practice day information, and the exercise information. The device also comprises an output unit which outputs the generated exercise plan to a notification device that notifies the user of the exercise plan. In addition, the exercise plan generation unit modifies the exercise information or the exercise plan, based on the result of the determination by the physical condition determination unit.

In the exercise support device according to this application example, the exercise plan generation unit generates an exercise plan generated using practice day information leading up to an event for a user, an exercise menu and event information. The exercise plan generation unit modifies the exercise menu or the exercise plan, based on the result of determination on the physical condition of the user found from pulse wave information. Thus, the user can obtain the exercise menu or the exercise plan modified based on the result of the determination on his/her own physical condition found from the pulse wave information, and can carry out effective training until the event, preventing overtraining and injuries or the like.

### APPLICATION EXAMPLE 2

In the exercise support device according to the application example, it is preferable that the event information includes at least one of a time and a date of the event, an identification of a type of competition to which the event belongs, and environment information.

According to this application example, detailed information about various conditions such as the number of days until the event for the user, the content of the competition, and the venue, elevation above sea level, weather and the like included in the environment information can be obtained in advance. Therefore, an exercise menu or an exercise plan based on this detailed information can be obtained.

### APPLICATION EXAMPLE 3

In the exercise support device according to the application example, it is preferable that the activity menu acquisition unit acquires the practice day information by at least one of an input from the user and an estimation operation based on stored past performance information of the user.

According to this application example, the practice day information can be easily acquired by an input from the user or by an estimation based on past performance information of the user.

### APPLICATION EXAMPLE 4

In the exercise support device according to the application example, it is preferable that the activity menu acquisition unit determines a value of each of a plurality of parameters of the exercise to be performed including an exercise time and an exercise intensity, based on at least one of the physical condition of the user and an environment where the exercise is performed, and the activity menu acquisition unit includes the determined values of the parameters in the exercise information.

According to this application example, an exercise menu is decided based on at least one of the physical condition of the user and the environment where the exercise is carried out. Therefore, an exercise menu including an exercise time and an exercise intensity can be decided in a way that suits the current state (physical condition) of the user.

### APPLICATION EXAMPLE 5

In the exercise support device according to the application example, it is preferable that the event is a physical competition, the acquired event information identifies the physical competition as the event, and the activity menu acquisition unit determines a suggested exercise to be performed on the practice day belonging to the same category of exercise used in the physical competition indicated in the event information, and includes the suggested exercise in the exercise information.

According to this application example, an exercise belonging to the same category as the athletic event included in the event information is suggested as the exercise menu. Therefore, the user can carry out an efficient exercise menu which is similar to the athletic event.

### APPLICATION EXAMPLE 6

In the exercise support device according to the application example, it is preferable that the exercise plan generation unit determines the practice day by calculating a degree of fatigue accumulated by the user in the case where the exercise to be performed is performed, using the pulse information, and setting a day when the degree of fatigue becomes a predetermined value or below the predetermined value, as the practice day.

According to this application example, the degree of fatigue accumulated in the case where the exercise menu is carried out is calculated based on the pulse wave information, and a day when the degree of fatigue of the user is predetermined value (preset threshold) or below is set as the practice day. Therefore, the user can carry out practice (training) on a day when the degree of fatigue is low, that is, on a day when the user is in good physical condition. Thus, the user can carry out effective practice (training) until the event, preventing overtraining and injuries or the like.

### APPLICATION EXAMPLE 7

In the exercise support device according to the application example, it is preferable that the pulse information is pulse rate variation information of the user, the physical condition determination unit determines a change in the physical condition of the user if the pulse rate variation information does not satisfy a predetermined condition, and the exercise plan generation unit modifies the exercise information or the exercise plan, based on a result of the determination by the physical condition determination unit.

According to this application example, pulse rate variation information of the user is used as the pulse wave information, and if the pulse rate variation information does not satisfy a predetermined condition (threshold), a change in the physical condition of the user is determined and the exercise menu or the exercise plan is modified accordingly. Therefore, the user can carry out effective practice (training) until the event, preventing overtraining and injuries or the like. As an example of the pulse rate variation information, HRV (heart rate variability) can be employed.

### APPLICATION EXAMPLE 8

In the exercise support device according to the application example, it is preferable that the predetermined condition is that the pulse rate variation information of the user is within a range between a standard deviation above an average value of the pulse rate variation information and a standard deviation below the average value of the pulse rate variation information.

According to this application example, a change in the physical condition of the user is determined, based on whether the pulse rate variation information of the user is within a range including the average value of the pulse rate variation information or not, as the predetermined condition (threshold). Therefore, the physical condition of the user, which changes constantly, can be determined, including its variations. Thus, the determination can be carried out with a higher degree of certainty.

### APPLICATION EXAMPLE 9

In the exercise support device according to the application example, it is preferable that the pulse information includes a first indicator which indicates a degree of variation of the pulse rate variation information measured when the user starts sleeping and a second indicator which indicates a degree of variation of the pulse rate variation information measured when the user ends sleeping. In addition, the physical condition determination unit evaluates the degree of fatigue of the user or a degree of recovery from fatigue of the user, based on a difference between the first indicator and the second indicator. Also, the exercise plan generation unit sets the exercise plan, based on a result of the evaluation on the degree of recovery from fatigue.

According to this application example, the degree of fatigue of the user or the degree of recovery from fatigue is evaluated, based on the difference between the first indicator and the second indicator, which indicate the degrees of variation of the pulse rate variation information measured at the start and end of sleep. The user carries out practice (training) according to an exercise plan that is set to be, for example, vigorous (hard) if the degree of recovery from fatigue is sufficient, or light (soft) if the degree of fatigue is high, based on the result of the evaluation. Therefore, the user can carry out effective practice (training) until the event, preventing overtraining and injuries or the like.

### APPLICATION EXAMPLE 10

An exercise support method performed by a processor in accordance with a program stored in a memory, include: acquiring event information about an event in which a user plans to participate; acquiring practice day information identifying the practice day on which the user will exercise before the event, and exercise information representing a value of a parameter of the exercise to be performed on the practice day; generating an exercise plan identifying the practice day and the value of the parameter of the exercise to be performed on the practice day, using the event information, the practice day information, and the exercise information; acquiring pulse information generated by a pulse sensor about a pulse of the user; determining a physical condition of the user based on the pulse information; modifying the exercise information or the exercise plan, based on a result of the determination in the determining step of the physical condition of the user; and outputting the generated or modified exercise plan to a notification device that notifies the user of the generated or modified exercise plan.

In the exercise support method according to this application example, an exercise plan is generated using practice day information leading up to an event for a user, an exercise menu and event information that are acquired. The physical condition of the user is determined based on pulse wave information of the user that is acquired, and the exercise menu or the exercise plan is modified, based on the result of the determination. Thus, the user can obtain the exercise menu or the exercise plan modified based on the result of the determination on his/her own physical condition found from the pulse wave information, and can carry out effective training until the event, preventing overtraining and injuries or the like.

### APPLICATION EXAMPLE 11

In the exercise support method according to the application example, it is preferable that the pulse information is pulse rate variation information of the user, in the determining of the physical condition of the user in the determining step, a change in the physical condition of the user is determined if the pulse rate variation information does not satisfy a predetermined condition, and in the modifying step for modifying the exercise plan, the exercise information or the exercise plan is modified, based on a result of the determination in the determining step.

According to this application example, pulse rate variation information of the user is used as the pulse wave information, and if the pulse rate variation information does not satisfy a predetermined condition (threshold), a change in the physical condition of the user is determined and the exercise menu or the exercise plan is modified accordingly. Therefore, the user can carry out effective practice (training) until the event, preventing overtraining and injuries or the like.

### APPLICATION EXAMPLE 12

An exercise support program according to this application example includes: acquiring practice day information leading up to an event for a user and an exercise menu; generating an exercise plan using event information, the practice day information, and the exercise menu; determining physical condition of the user based on pulse wave information of the user that is acquired; and modifying the exercise menu or the exercise plan, based on a result of the determination.

In the exercise support program according to this application example, an exercise plan is generated using practice day information leading up to an event for a user, an exercise menu and event information that are acquired. The physical condition of the user is determined based on pulse wave information of the user that is acquired, and the exercise menu or the exercise plan is modified, based on the result of the determination. Thus, the user can obtain the exercise menu or the exercise plan modified based on the result of the determination on his/her own physical condition found from the pulse wave information, and can carry out effective training until the event, preventing overtraining and injuries or the like.

### APPLICATION EXAMPLE 13

In the exercise support program according to the application example, it is preferable that the pulse wave information is pulse rate variation information of the user, that a change in the physical condition is determined if the pulse rate variation information does not satisfy a predetermined condition, and that the exercise menu or the exercise plan is modified, based on a result of the determination.

According to this application example, pulse rate variation information of the user is used as the pulse wave information, and if the pulse rate variation information does not satisfy a predetermined condition (threshold), a change in the physical condition of the user is determined and the exercise menu or the exercise plan is modified accordingly. Therefore, the user can carry out effective practice (training) until the event, preventing overtraining and injuries or the like.

### APPLICATION EXAMPLE 14

An exercise support system according to this application example includes: a detection device which detects pulse wave information of a user; the exercise support device according to one of the foregoing application examples; and a notification unit which notifies the user of an exercise menu or an exercise plan modified based on a result of determination on physical condition of the user from the pulse wave information, in the exercise support device.

In the exercise support system according to this application example, the exercise support device processes pulse wave information of the user detected by the detection device, and the notification unit can notify the user of an exercise menu or an exercise plan modified based on the result of determination on the physical condition of the user. Thus, the user can obtain the exercise menu or the exercise plan modified based on the result of the determination on his/her own physical condition found from the pulse wave information, and can carry out effective training until the event, preventing overtraining and injuries or the like.

### APPLICATION EXAMPLE 15

In the exercise support method according to the application example, it is preferable that the event information includes at least one of a time and a date of the event, an identification of a type of competition to which the event belongs, and environment information.

### APPLICATION EXAMPLE 16

In the exercise support method according to the application example, it is preferable that the practice day information is acquired by at least one of an input from the user and an estimation operation based on stored past performance information of the user.

### APPLICATION EXAMPLE 17

In the exercise support method according to the application example, it is preferable that the method further comprises determining a value of each of a plurality of parameters of the exercise to be performed including an exercise time and an exercise intensity, based on at least one of the physical condition of the user and an environment where the exercise is performed, and including the determined value of each of the plurality of parameters in the exercise information.

### APPLICATION EXAMPLE 18

In the exercise support method according to the application example, it is preferable that the event is a physical competition and the acquired event information identifies the physical competition to which the event belongs, wherein the method further comprises determining a suggested exercise to be performed on the practice day belonging to the same category of exercise used in the physical competition identified in the event information, and including the suggested exercise in the exercise information.

### APPLICATION EXAMPLE 19

In the exercise support method according to the application example, it is preferable that the method further includes step of determining the practice day by calculating degree of fatigue accumulated by the user in the case where the exercise to be performed is performed, using the pulse information, and setting a day when the degree of fatigue becomes a predetermined value or below the predetermined value as the practice day.

### APPLICATION EXAMPLE 20

An exercise support system according to this example includes a memory storing a program, and a processor. The processor, when performing the program, functions as: a pulse information acquisition unit which acquires pulse information generated by a pulse sensor about a pulse of the user; a physical condition determination unit which determines a physical condition of the user based on the pulse information; an event information acquisition unit which acquires event information about an event in which the user plans to participate; an activity menu acquisition unit which acquires practice day information identifying the practice day on which the user will exercise before the event and exercise information representing a value of a parameter of the exercise to be performed on the practice day; an exercise plan generation unit which generates an exercise plan identifying the practice day and the value of the parameter of the exercise to be performed on the practice day, using a result of the determination on the physical condition of the user by the physical condition determination unit, the event information, the practice day information, and the exercise information. The system also includes a notification device which notifies the user of at least one of the exercise information and the exercise plan.

### APPLICATION EXAMPLE 21

In the exercise support system according to the application example, it is preferable that the exercise plan generation unit modifies the exercise information or the exercise plan, based on a result of the determination by the physical condition determination unit.

### APPLICATION EXAMPLE 22

In the exercise support system according to the application example, it is preferable that the exercise plan generation unit determines the practice day by calculating a degree of fatigue accumulated by the user in the case where the exercise to be performed is performed, using the pulse information, and setting a day when the degree of fatigue becomes a predetermined value or below the predetermined value, as the practice day.

### APPLICATION EXAMPLE 23

In the exercise support system according to the application example, it is preferable that the pulse information is pulse rate variation information of the user, the physical condition determination unit determines a change in the physical condition of the user if the pulse rate variation information does not satisfy a predetermined condition, and the exercise plan generation unit modifies the exercise information or the exercise plan, based on a result of the determination by the physical condition determination unit.

### APPLICATION EXAMPLE 24

An exercise support system according to this example includes a memory storing a program, and a processor. The processor, when performing the program, functions as: an exercise plan generation unit which generates an exercise plan identifying i) a practice day on which a user will perform an exercise before an event in which the user will participate, and ii) a value of a parameter of the exercise to be performed on the practice day, the exercise plan generation unit identifying the practice day by identifying the day on which the event will occur from acquired event information, acquiring exercise information representing a value of a parameter of the exercise to be performed, calculating the user's accumulated degree of fatigue in the event the user performs the exercise to be performed using pulse information about the user's pulse generated by a pulse sensor, and selecting a day before the event in which the user's accumulated fatigue becomes no greater than a predetermined value. The support device also includes an output unit that outputs the generated exercise plan to a notification device that notifies the user of the exercise plan.

### APPLICATION EXAMPLE 25

In the exercise support system according to the application example, it is preferable that the exercise plan generation unit modifies the exercise information or the exercise plan, based on the result of the determination by the physical condition determination unit, and the output unit outputs the modified exercise information or the modified exercise plan.

### APPLICATION EXAMPLE 26

An exercise support system according to this example includes a memory storing a program, and a processor. The processor, when performing the program, functions as: an exercise plan generation unit which generates an exercise plan identifying the practice day on which a user will exercise before an event in which the user participates and a value of a parameter of an exercise to be performed on the practice day included in exercise information received by the exercise plan generation unit; a pulse information acquiring unit which acquires pulse information generated by a pulse sensor about a pulse of the user; and a physical condition determination unit which determines a physical condition of the user based on the pulse information. In addition, the exercise plan generation unit modifies the exercise information or the exercise plan, based on a result of the determination by the physical condition determination unit of the physical condition of the user. Also, the exercise support system also includes an outputting unit which outputs the generated or modified exercise plan to a notification device that notifies the user of the generated or modified exercise plan.

### BRIEF DESCRIPTION OF THE DRAWINGS

The invention will be described with reference to the accompanying drawings, wherein like numbers reference like elements.

FIG. 1 is a schematic configuration diagram showing an outline of an exercise support system.

FIG. 2 is an external view showing a schematic configuration of a wearable device used in the exercise support system.

FIG. 3 is an external view showing an example of wearing the wearable device.

FIG. 4 is a cross-sectional view showing the configuration of the wearable device.

FIG. 5 is a block diagram showing a configuration example of an exercise support device used in the exercise support system.

FIG. 6 shows a process of recovery from fatigue (degree of fatigue) and the correlation between fatigue (degree of fatigue) and time elapsed from training.

FIG. 7 explains HRV (heart rate variability).

FIG. 8A shows the correlation between performance and time elapsed at the time of performance drop.

FIG. 8B is a graph showing HRV (heart rate variability) in the state of a zone P in FIG. 8A.

FIG. 9A shows the correlation between performance and time elapsed at the time of performance rise.

FIG. 9B is a graph showing HRV (heat rate variability) in the state of a zone Q in FIG. 9A.

FIG. 10 is a flowchart showing Example 1 of an exercise support method.

FIG. 11 is a graph showing physical condition determination example 1 based on HRV (heat rate variability).

FIG. 12 is a flowchart showing Example 2 of the exercise support method.

FIG. 13 shows an example of setting practice days.

FIG. 14 is a flowchart showing Example 3 of the exercise support method.

FIG. 15A is a first graph for explaining physical condition determination example 2 based on HRV (heat rate variability).

FIG. 15B is a second graph for explaining physical condition determination example 2 based on HRV (heat rate variability).

### DESCRIPTION OF EXEMPLARY EMBODIMENTS

Hereinafter, embodiments of an exercise support system (exercise support device), an exercise support method, and an exercise support program according to the invention will be described. The embodiments described below should not unduly limit the content of the invention described in the appended claims. Not all of the configurations described in each embodiment are necessarily essential elements of the invention.

### 1. Technique According to Present Embodiment

First, an embodiment of an exercise support system (exercise support device) according to the invention will be described. In the description below, as an example of a detection device used in the exercise support system, for example, a wearable device which has a pulse wave sensor and a body motion sensor and is worn around the user's wrist is used. In the wearable device used in the exercise support system, a pulse wave sensor as a biological sensor which acquires pulse wave information as biological information is used. With this pulse wave sensor, pulse wave information such as pulse rate and heartbeat interval (RRI: R-R interval) can be acquired.

As the pulse wave sensor, for example, a photoelectric sensor is used. In this case, a technique such as detecting reflected light or transmitted light of light cast on a living body with the photoelectric sensor is conceivable. The amount of light absorbed by the living body and the amount of light reflected by the living body, of the light cast thereon, vary according to the amount of blood flow in the blood vessels. Therefore, sensor information detected by the photoelectric sensor is a signal corresponding to the amount of blood flow or the like. By analyzing this signal, information about pulsation can be acquired. However, the pulse wave sensor is not limited to the photoelectric sensor. Other sensors such as electrocardiograph and ultrasonic sensor may be used. The body motion sensor is a sensor which detects body motions of the user. As the body motion sensor, an acceleration sensor, angular velocity sensor or the like may be used. However, other sensors may be used as well.

As an example of the wearable device, a wearable device which is worn around the wrist and has a pulse wave sensor is used. However, the wearable device according to each embodiment may be worn at other parts of the user such as the neck or ankle. The exercise support device and the exercise support system according to each embodiment may include a biological sensor other than the photoelectric sensor.

In the exercise support system including the photoelectric sensor (pulse wave sensor), it is necessary to receive necessary light and block unnecessary light. In the example of the pulse wave sensor, reflected light including a pulse wave component reflected by a subject as a measurement target object (particularly a part including a measurement target blood vessel) is received as intense light, whereas other lights are noise components and therefore blocked.

### 2. Exercise Support System

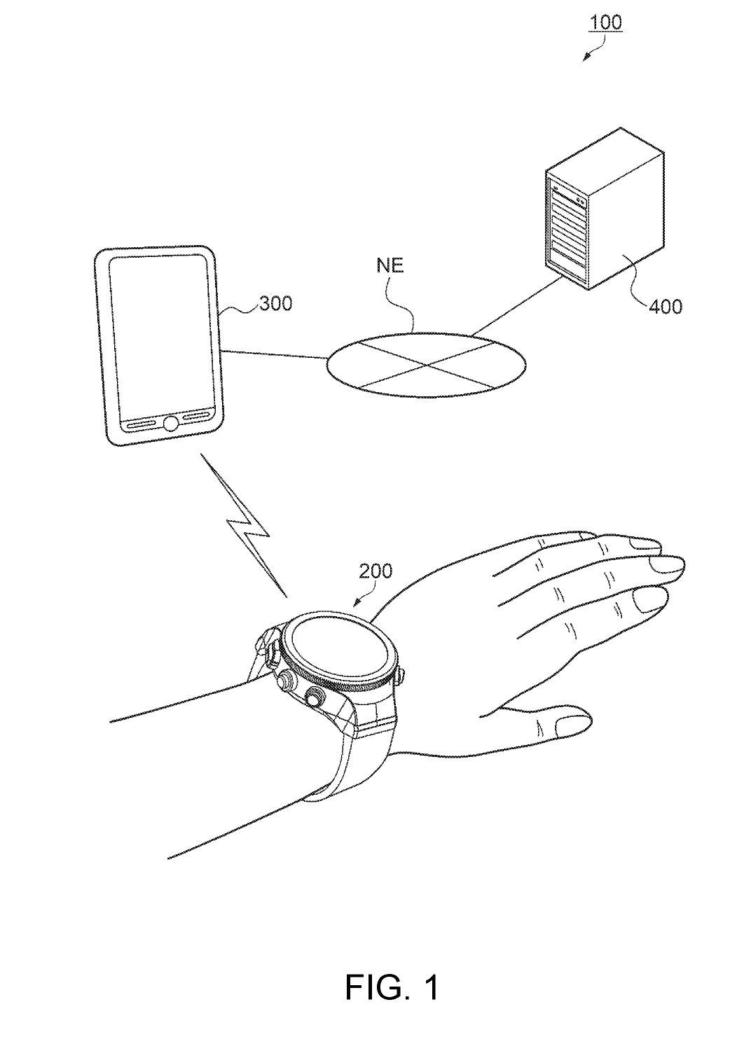

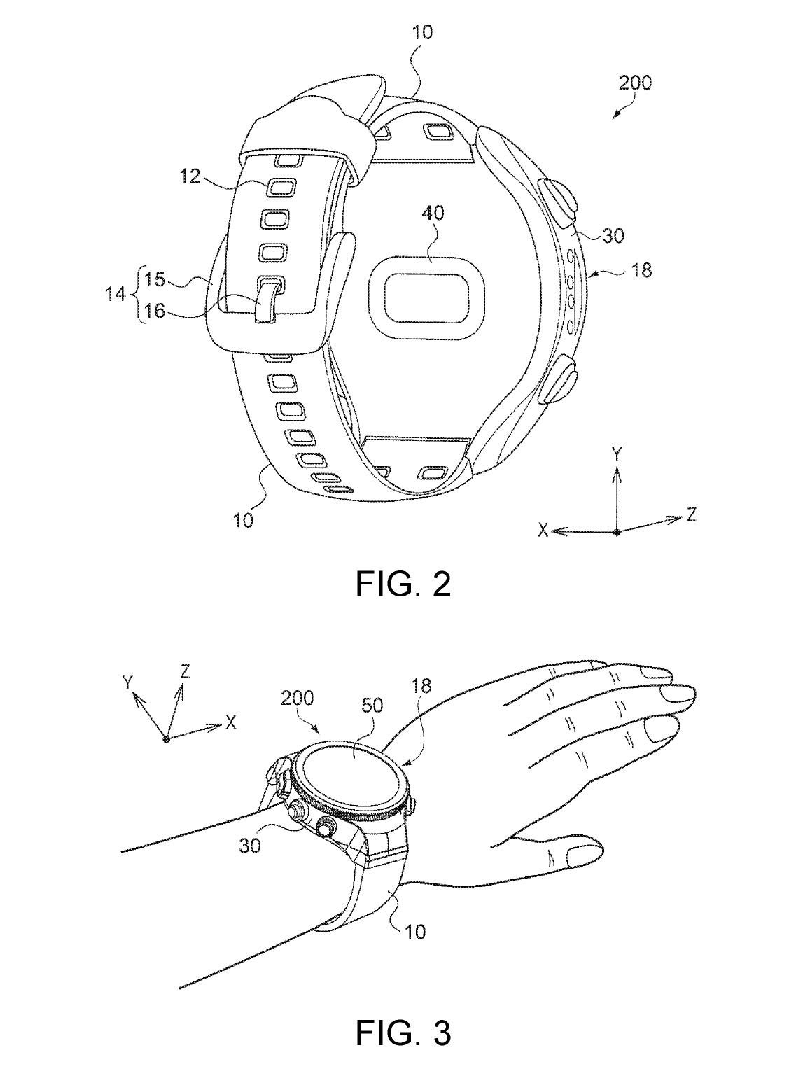

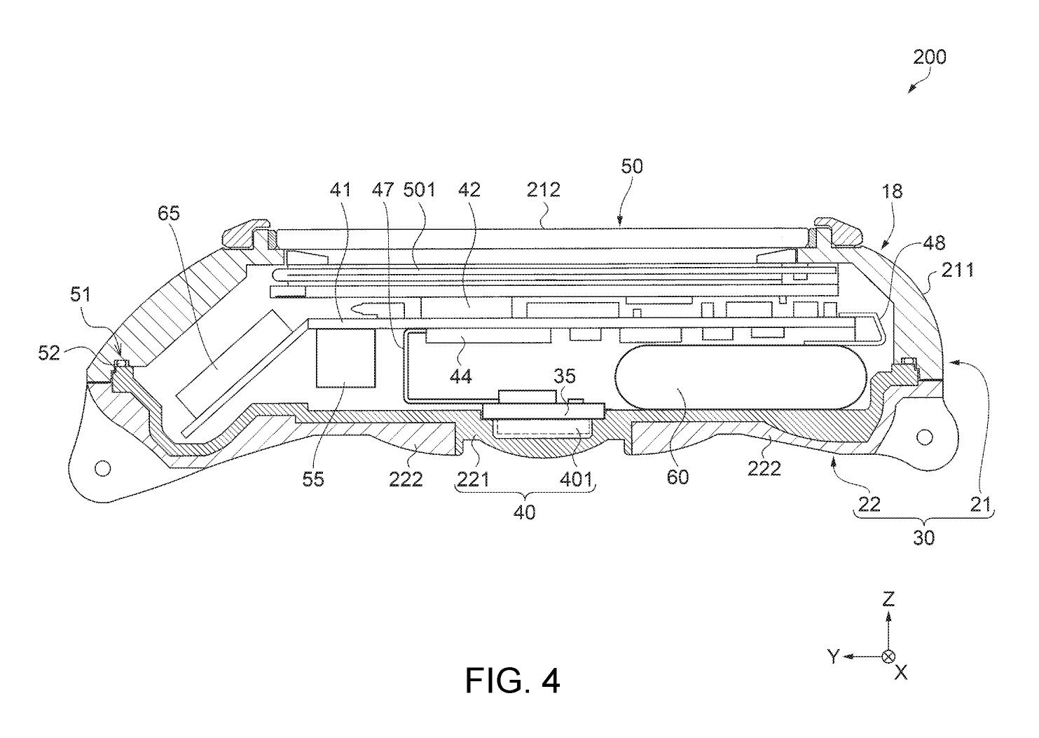

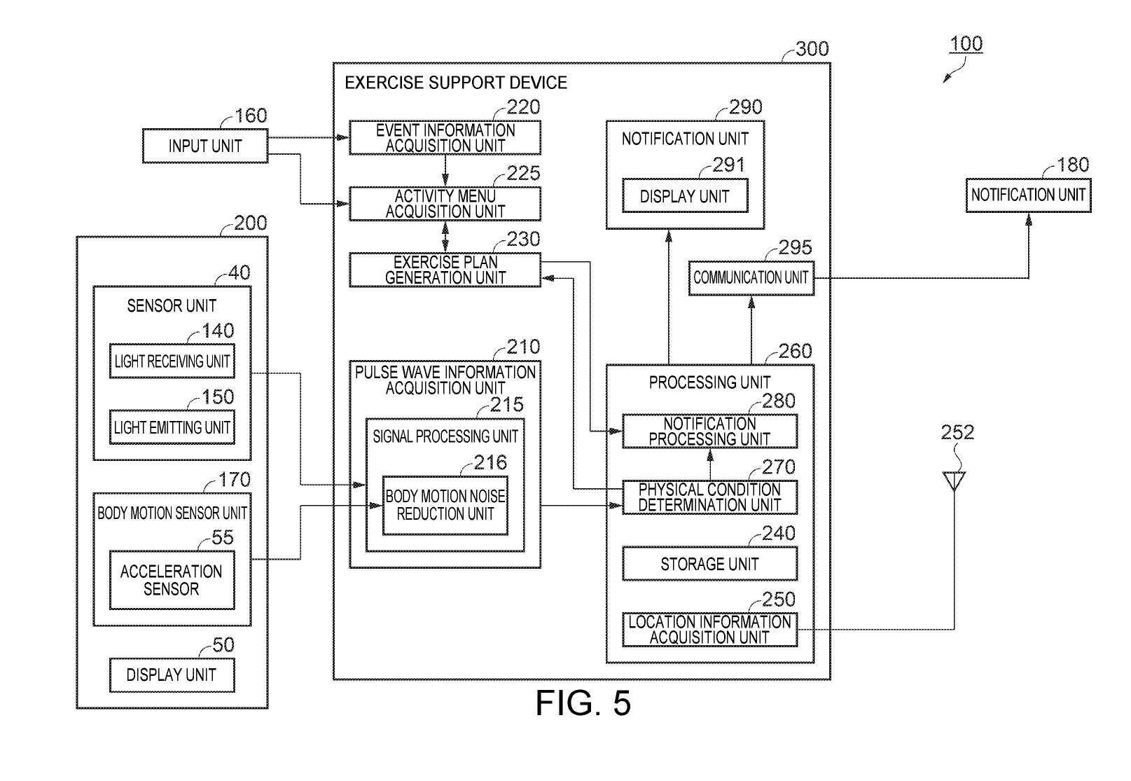

Next, the configuration of the exercise support system and the exercise support device according to the embodiment will be described, referring to FIGS. 1, 2, 3, 4, and 5. FIG. 1 is a schematic configuration diagram showing an outline of the exercise support system. FIG. 2 is an external view showing a schematic configuration of the wearable device used in the exercise support system. FIG. 3 is an external view showing an example of wearing the wearable device. FIG. 4 is a cross-sectional view showing the configuration of the wearable device. FIG. 5 is a block diagram showing a configuration example of the exercise support device used in the exercise support system.

An exercise support system 100 according to the embodiment includes a wearable device 200 as a detection device using a pulse sensor as a biological sensor (photoelectric sensor) which is a photoelectric sensor, a mobile terminal device 300, and an information processing device 400 connected to the mobile terminal device 300 via a network NE, as shown in FIG. 1.

The mobile terminal device 300 can be made up of, for example, a smartphone or tablet terminal device. The mobile terminal device 300 is connected to the wearable device 200 using a pulse sensor as a biological sensor (photoelectric sensor) which is a photoelectric sensor), via short-range wireless communication or wired communication (not illustrated) or the like. The mobile terminal device 300 can be connected to the information processing device 400 such as a PC (personal computer) or server system via the network NE. As the network NE in this example, various networks such as WAN (wide area network), LAN (local area network), and short-range wireless communication can be used. In this case, the information processing device 400 is realized as a processing and storing unit which receives pulse wave information and body motion information measured by the wearable device 200 via the network NE and stores the pulse wave information and the body motion information.

The wearable device 200 only needs to be able to communicate with the mobile terminal device 300 and need not be connected directly to the network NE. Therefore, the configuration of the wearable device 200 can be simplified. However, the exercise support system 100 can also employ a modified configuration in which the wearable device 200 and the information processing device 400 are directly connected, omitting the mobile terminal device 300. Also, while the exercise support system 100 is a system where exercise support is carried out through communication or the like between the wearable device 200, the mobile terminal device 300, and the information processing device 400, exercise support may also be carried out by a configuration in which the functions of the mobile terminal device 300 and the information processing device 400, described later, are realized by a single device, that is, by an exercise support device.

The exercise support system 100 is not limited to being realized by the information processing device 400. For example, the exercise support system 100 may be realized by the mobile terminal device 300. For example, the mobile terminal device 300, such as a smartphone, often has limits on its processing ability, storage area, and battery capacity, compared with a server system. However, considering the recent improvement in performance, it may be possible to secure sufficient processing ability or the like. Therefore, if requirements for processing ability or the like are satisfied, the mobile terminal device 300 can be used as the exercise support system 100 according to the embodiment.

The exercise support system 100 according to the embodiment is not limited to being realized by a single device. For example, the exercise support system 100 may include two or more of the wearable device 200, the mobile terminal device 300 as an exercise support device, and the information processing device 400. In this case, the processing executed in the exercise support system 100 may be executed by one of these devices, or may be distributed among a plurality of devices. Also, the exercise support system 100 according to the embodiment may include a device that is different from the wearable device 200 as a detection device, the mobile terminal device 300, and the information processing device 400.

Moreover, if improvement in the performance of the terminal or the way of using the system or the like is considered, the exercise support system 100 (mobile terminal device 300) according to the embodiment can be realized by the wearable device 200.

Also, the processing by each part of the exercise support system 100 according to the embodiment can be realized by a program. That is, the technique according to the embodiment can be applied to a program which causes a computer to execute processing of generating an exercise plan generated using practice day information leading up to an event for the user, an exercise menu, and event information, based on pulse wave information of the user and the event information that are acquired, and modifying the exercise menu or the exercise plan, based on the result of determination on physical condition of the user found from the pulse wave information.

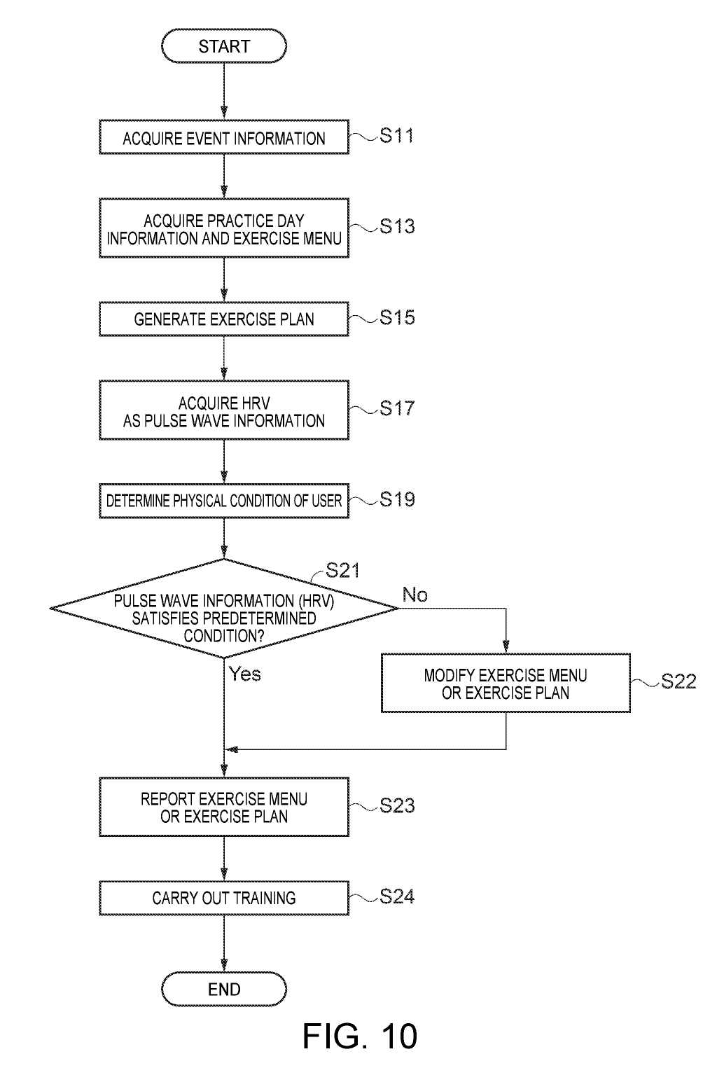

With this program, for example, the following computation and notification processing can be carried out. More specifically, the program according to the embodiment can cause a computer to execute each step shown in FIGS. 10, 12, and 14, described later.

1) Practice day information is acquired by at least one of an input from the user and an estimation based on past performance information of the user.

2) An exercise menu including an exercise time and an exercise intensity is decided, based on at least one of the physical condition of the user and the environment where the exercise is carried out.

3) An exercise belonging to the same category as the athletic event included in the event information is suggested as an exercise menu.

4) As an exercise plan, the degree of fatigue accumulated in the case where the exercise menu is carried out is calculated using pulse wave information, and a day when the degree of fatigue becomes a predetermined value or below is set as a practice day.

5) HRV (heart rate variability) as pulse rate variation information of the user is referred to as the degree of fatigue, and if the pulse rate variation information (HRV) does not satisfy a predetermined condition, a change in the physical condition of the user is determined and the exercise menu or the exercise plan is modified.

The exercise support system 100 also includes a memory which stores information (for example, a program and various data), and a processor which operates based on the information stored in the memory. In the processor, for example, the functions of individual parts may be realized by individual pieces of hardware, or the functions of individual parts may be realized by integrated hardware. The processor may be, for example, a CPU. However, the processor is not limited to the CPU. Various processors such as GPU (graphics processing unit) or DSP (digital signal processor) can be used. The processor may also be an ASIC-based hardware circuit. The memory may be, for example, a semiconductor memory such as SRAM (static random access memory) or DRAM (dynamic random access memory), a register, a magnetic storage device such as hard disk device, or an optical storage device such as optical disk device. For example, the memory stores computer-readable commands, and the functions of each part of the exercise support system 100 are realized by the processor executing these commands. The commands in this case may be commands in a command set which forms a program, or may be commands which instruct the hardware circuit of the processor to carry out operations.

The wearable device 200 is worn at a predetermined part of the user's body (for example, a measurement target object such as the wrist), as shown in FIGS. 2, 3, and 4, and detects pulse wave information or the like. The wearable device 200 has a device main body 18 which includes a case section 30 and comes in tight contact with the user so as to detect pulse wave information or the like, and a pair of strap sections 10 which is attached to the device main body 18 and is for the user to wear the device main body 18, as shown in FIG. 2. The device main body 18 including the case section 30 is provided with a display unit 50 and a sensor unit 40. The strap sections 10 are provided with a fitting hole 12 and a buckle 14. The buckle 14 is made up of a buckle frame 15 and an engagement part (protruding rod) 16.

In the description of the wearable device 200 below, the side of the device main body 18 situated on the side of the measurement target object (subject) when the device main body 18 is installed on the user is referred to as a “back side or back surface side”, and the display surface side of the device main body 18, which is the opposite side, is referred to as a “front side or front surface side”. The “target object” of measurement may also be referred to as a “subject”. A coordinate system is set, using the case section 30 of the wearable device 200 as a point of reference. A direction which intersects with the display surface of the display unit 50 and heads from the back surface toward the front surface in the case where the display surface side of the display unit 50 is regarded as the surface is defined as a positive Z-axis direction. Alternatively, a direction heading from the sensor unit 40 toward the display unit 50, or a direction away from the case section 30 in the direction of a normal line to the display surface of the display unit 50 may be defined as a positive Z-axis direction. In the state where the wearable device 200 is worn on the subject, the positive Z-axis direction is equivalent to a direction heading from the subject toward the case section 30. Two axes orthogonal to the Z-axis are defined as X and Y axes. Particularly a direction in which the strap sections 10 are attached to the case section 30 is set to the Y-axis.

FIG. 2 is a perspective view of the wearable device 200 in the state where the strap sections 10 are fixed using the fitting hole 12 and the engagement part 16, as viewed from the −Z-axis direction, which is the direction on the side of the strap sections 10 (the side of the surface that comes on the subject side in the wearing state, of the surfaces of the case section 30). In the wearable device 200, a plurality of fitting holes 12 is provided in the strap sections 10. By having the engagement part 16 of the buckle 14 inserted into one of the fitting holes 12, the wearable device 200 is installed on the user. The plurality of fitting holes 12 is provided along the longitudinal direction of the strap sections 10.

The device main body 18 has the case section 30 including a top case 21 and a bottom case 22, as shown in FIG. 4. The bottom case 22 is situated on the side of the measurement target object when the device main body 18 is installed on the user. The top case 21 is arranged on the side opposite to the measurement target object (surface side), in contrast to the bottom case 22. A detection window 221 (see FIG. 4) is provided on the back side of the bottom case 22. The sensor unit 40 is provided at the position corresponding to the detection window 221.

FIG. 2 shows an example where the use of a biological sensor (photoelectric sensor 401 (see FIG. 4) as pulse wave sensor for acquiring pulse wave information) is assumed and where the sensor unit 40 is provided on the side that comes on the subject side when the wearable device 200 is installed. However, the position where the biological sensor is provided is not limited to the position illustrated in FIG. 2. For example, the biological sensor may be provided inside the case section 30.

FIG. 3 shows the wearable device 200 in the state of being worn by the user, as viewed from the side where the display unit 50 is provided (Z-axis direction). As shown in FIG. 3, the wearable device 200 according to the embodiment has the display unit 50 at a position equivalent to the face of an ordinary wristwatch or a position where numerals and icons can be visually recognized. In the wearing state of the wearable device 200, the side of the bottom case 22 (see FIG. 4), of the case section 30, is in tight contact with the subject, and the display unit 50 is at a position where visual recognition by the user is easy.

Next, an example of the detailed cross-sectional structure of the device main body 18 of the wearable device 200 will be described, referring to FIG. 4. As shown in FIG. 4, the device main body 18 includes a module board 35, the sensor unit 40 connected to the module board 35, a circuit board 41, a panel frame 42, a circuit case 44, an LCD 501 forming the display unit 50, an acceleration sensor 55 as an example of the body motion sensor, a secondary battery 60, and a GPS antenna 65, in addition to the top case 21 and the bottom case 22. However, the configuration of the wearable device 200 is not limited to the configuration shown in FIG. 4. It is possible to add another configuration or omit a part of the configuration.

The top case 21 may include a trunk part 211 and a glass plate 212. In this case, the trunk part 211 and the glass plate 212 may be used as outer walls for protecting the internal structure and may be configured in such a way that the user can view, through the glass plate 212, a display on the display unit 50 such as a liquid crystal display (hereinafter the LCD 501) provided directly below the glass plate 212. That is, in the embodiment, various kinds of information such as extracted biological information, information indicating an exercise state, or time information, may be displayed using the LCD 501, and this display may be presented to the user from the side of the top case 21. In this example, the top part of the wearable device 200 is realized by the glass plate 212. However, the top part can be formed of materials other than glass, such as a transparent plastic, provided that it is a transparent member through which the LCD 501 can be viewed and which has enough strength to be able to protect the components included inside the case section 30 such as the LCD 501.

The bottom case 22 is provided with the detection window 221 and a bank part 222. The bank part 222 protrudes in a direction from the bottom case 22 toward the subject. The detection window 221 is provided on the bank part 222. The sensor unit 40 is provided at the position corresponding to the detection window 221. The detection window 221 is configured to transmit light. Light emitted from a light emitting unit 150 (see FIG. 5) included in the sensor unit 40 is transmitted through the detection window 221 and cast on the subject (measurement target object). The detection window 221 has a convex part protruding from the sensor unit 40 toward the subject. A groove part is provided between the convex part of the detection window 221 and the bank part 222. With this configuration, stable measurement of pulse waves can be realized even during an exercise, for example, as described in detail in JP-A-2014-180291. Also, reflected light from the subject is transmitted through the detection window 221 and receives by a light receiving unit 140 (see FIG. 5) of the sensor unit 40. That is, the provision of the detection window 221 enables detection of biological information using the photoelectric sensor. The sensor unit 40 is connected to the module board 35. The module board 35 is electrically connected to the circuit board 41, for example, using a flexible board 47 or the like.

On one surface of the circuit board 41, the panel frame 42 for guiding the display panel such as the LCD 501 is arranged. On the other surface of the circuit board 41, the circuit case 44 for guiding the secondary battery 60 or the like is arranged. On the circuit board 41, elements which form a circuit for driving the sensor unit 40 to measure pulse rate, a circuit for driving the LCD 501, and a circuit for controlling each circuit or the like, are mounted. The circuit board 41 is made electrically continuous to an electrode of the LCD 501 via a connector, not illustrated. The LCD 501 displays pulse rate measurement data such as pulse rate, time information such as the current time, and the like, according to each mode.

The circuit case 44 accommodates the secondary battery 60 (lithium secondary battery), which is rechargeable. The secondary battery 60 has its two pole terminals connected to the circuit board 41 via a connection board 48 or the like, and supplies electricity to a circuit which controls the power source. This electricity is converted into a predetermined voltage by this circuit and is supplied to each circuit, and thus causes each circuit to operate, such as the circuit for driving the sensor unit 40 to measure pulse rate, the circuit for driving the LCD 501, and the circuit for controlling each circuit. The charging of the secondary battery 60 is carried out via a pair of charging terminals which is electrically continuous to the circuit board 41 via an electrical conduction member (not illustrated) such as a coil spring. While an example of using the secondary battery 60 as the battery is described here, a primary battery which does not need charging may be used as the battery.

As shown in FIG. 4, the detection window 221 may be formed to extend to a sealing part 51 provided at the connection part between the top case 21 and the bottom case 22. Here, the sealing part 51 may be provided with a packing 52 which seals the interior of the case section 30 from outside. The packing 52 is provided at the connection part between the top case 21 and the bottom case 22 and is configured to seal the interior of the case section 30 from outside.

### 3. Exercise Support Device

FIG. 5 shows a detail configuration example of the mobile terminal device 300 as an exercise support device according to the embodiment. The mobile terminal device 300 as an exercise support device includes: a pulse wave information acquisition unit 210 which acquires pulse wave information of the user; an event information acquisition unit 220 which acquires event information about an event for the user; an activity menu acquisition unit 225 which acquires practice day information leading up to the event and an exercise menu; an exercise plan generation unit 230 which generates an exercise plan using the event information, the practice day information, and the exercise menu; a processing unit 260 including at least a physical condition determination unit 270 which determines the physical condition of the user based on the pulse wave information; a notification unit 290 which notifies the user of information processed by the processing unit 260; and a communication unit 295 which carries out communication processing to and from outside, as shown in FIG. 5.

However, the mobile terminal device 300 is not limited to the configuration of FIG. 5. Various modifications can be made such as omitting a part of the components or adding another component. For example, the mobile terminal device 300 may include an input unit 160 or may include the sensor unit 40 or a body motion sensor unit 170 included in the wearable device 200.

The pulse wave information acquisition unit 210 acquires pulse wave information and body motion information of the user detected by the sensor unit 40 and the body motion sensor unit 170 included in the wearable device 200. The pulse wave information acquisition unit 210 includes a signal processing unit 215 which processes a signal (pulse wave information) detected by the sensor unit 40 and a signal (body motion information) detected by the body motion sensor unit 170.

The pulse wave information acquisition unit 210 carries out computation processing related to pulse wave information, for example, pulsation information or HRV (heart rate variability) (hereinafter also referred to simply as “HRV”) as pulse rate variation information, based on a signal or the like from the signal processing unit 215. The pulse wave information acquisition unit 210 transmits the result of the computation processing to the processing unit 260 (physical condition determination unit 270) as pulse wave information.

The pulsation information in this case is, for example, information of pulse rate or the like. Specifically, for example, the pulse wave information acquisition unit 210 carries out frequency analysis processing such as FFT on a pulse wave detection signal after noise reduction processing by a body motion noise reduction unit 216, thus obtains a spectrum, and carries out processing of determining a representative frequency in the resulting spectrum, as the frequency of heartbeat. The resulting frequency multiplied by 60 is a pulse rate (heart rate) which is commonly used. The HRV is an indicator indicating heart rate variability. The HRV will be described in detail later.

The signal processing unit 215 is configured to carry out various kinds of signal processing (filter processing and the like), and for example, carries out signal processing on a pulse wave detection signal from the sensor unit 40 and a body motion detection signal from the body motion sensor unit 170. For example, the signal processing unit 215 includes the body motion noise reduction unit 216. The body motion noise reduction unit 216 carries out processing of reducing (eliminating) a body motion noise which is a noise caused by a body motion, from the pulse wave detection signal, based on the body motion detection signal from the body motion sensor unit 170. Specifically, for example, the body motion noise reduction unit 216 carries out noise reduction processing using an adaptive filter or the like.

The sensor unit 40 is configured to detect pulse waves or the like, and includes the light receiving unit 140 and the light emitting unit 150. A pulse wave sensor (photoelectric sensor) is realized by the light receiving unit 140 and the light emitting unit 150 or the like. The sensor unit 40 outputs a signal detected by the pulse wave sensor, as a pulse wave detection signal. For example, a photoelectric sensor is used as the sensor unit 40. In this case, a technique such as detecting, with the light receiving unit 140, reflected light or transmitted light of light cast on a living body (user's wrist) from the light emitting unit 150 may be employed. With such a technique, since the amount of light absorbed by the living body and the amount of light reflected by the living body, of the light cast thereon, vary according to the amount of blood flow in blood vessels, sensor information detected by the photoelectric sensor is a signal corresponding to the amount of blood flow or the like and therefore information about pulsation can be acquired by analyzing this signal. However, the pulse wave sensor is not limited to the photoelectric sensor. Other sensors such as electrocardiograph and ultrasonic sensor may be used.

The body motion sensor unit 170 is a sensor which detects body motions of the user, and outputs a body motion detection signal which is a signal changing with body motions. The body motion sensor unit 170 includes, for example, the acceleration sensor 55 as the body motion sensor. The body motion sensor unit 170 may also have an angular velocity sensor, a pressure sensor, and a gyro sensor or the like, as the body motion sensor.

The event information acquisition unit 220 acquires event information about an event for the user. Here, the event information includes at least one of, for example, the time and date of a competition in which the user is going to participate, an athletic event (content of competition), and environment information (elevation above sea level, ups and downs, climate or the like at the venue). The event information can be acquired when the user inputs this information to the input unit 160 or acquired as network information via the network NE (see FIG. 1).

The activity menu acquisition unit 225 acquires practice day information leading up to the event for the user and an exercise menu. The practice day information acquired by the activity menu acquisition unit 225 is acquired by at least one of an input from the user and an estimation based on past performance information of the user stored in a storage unit 240. Thus, the practice day information can be easily acquired by an input from the user and by an estimation based on the past performance information of the user.

The exercise menu including the exercise time and the exercise intensity acquired by the activity menu acquisition unit 225 can be decided, based on at least one of the physical condition of the user and the environment where an exercise is carried out. Thus, by using as a factor at least one of the physical condition of the user and the environment where an exercise is carried out, an exercise menu including an exercise time and an exercise intensity can be decided according to the current state of the user.

The activity menu acquisition unit 225 can suggest an exercise belonging to the same category as the athletic event included in the acquired event information, as the exercise menu. Thus, since an exercise belonging to the same category as the athletic event included in the event information is suggested as the exercise menu by the activity menu acquisition unit 225, the user can carry out an efficient exercise menu of a kind closer to the athletic event.

The exercise plan generation unit 230 generates an exercise plan using the acquired event information of the event for the user, the practice day information, and the exercise menu suggested by the activity menu acquisition unit 225. The exercise plan generation unit 230 can also modify the exercise menu or the exercise plan, based on the result of determination by the physical condition determination unit 270, described later.

It is preferable that, as an exercise plan, the exercise plan generation unit 230 calculates the degree of fatigue accumulated in the case where the foregoing exercise menu is carried out, using HRV (heart rate variability) as the pulse rate variation information of the user obtained as a kind of pulse wave information, and sets a day when the degree of fatigue becomes a predetermined value (preset threshold) or below, as a practice day. Thus, since the degree of fatigue accumulated in the case where the exercise menu is carried out is calculated based on the pulse wave information (HRV) and a day when the degree of fatigue of the user becomes a predetermined value or below is set as a practice day, the user can carry out practice (training) on a day when the degree of fatigue is low, that is, when the user is in good physical condition. Thus, the user can carry out effective practice (training) until the event, preventing overtraining and injuries or the like.

If the HRV does not satisfy the predetermined condition (threshold), the exercise plan generation unit 230 can modify the suggested exercise menu or the exercise plan, based on the result of determination of a change in the physical condition by the physical condition determination unit 270, described later. Thus, since the HRV of the user is used as the pulse wave information and if the HRV does not satisfy the predetermined condition (threshold), a change in the physical condition of the user is determined and the exercise menu or the exercise plan is modified accordingly, the user can carry out effective practice (training) until the event, preventing overtraining and injuries or the like.

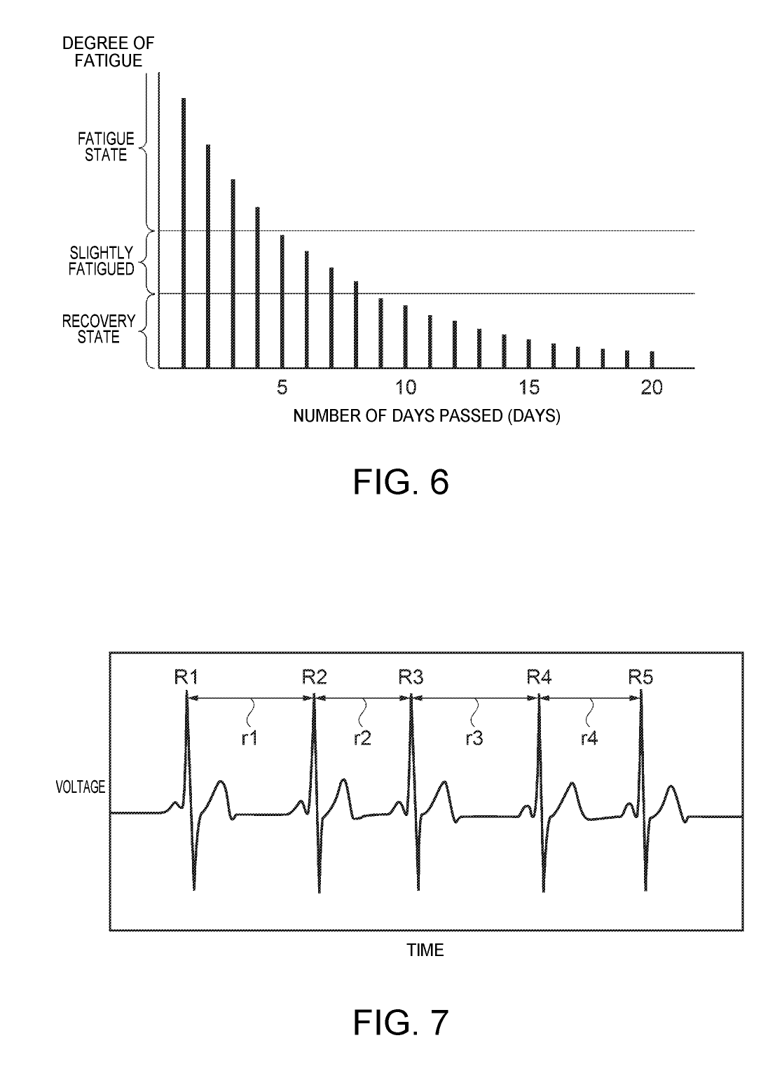

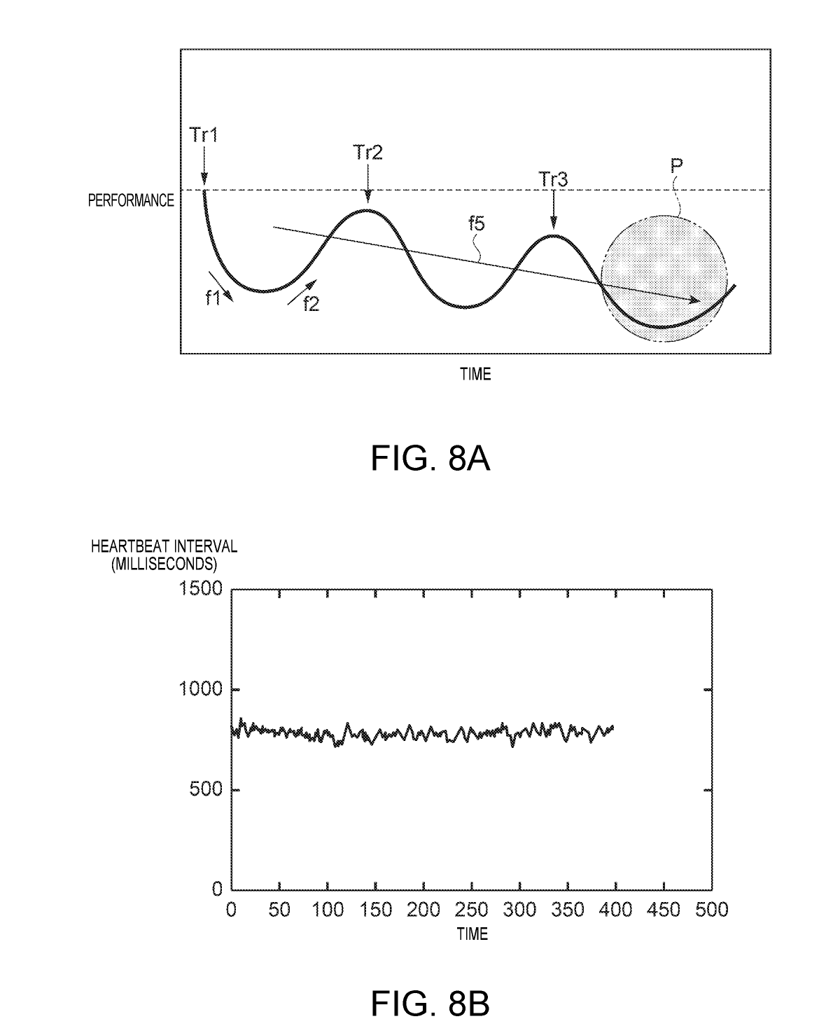

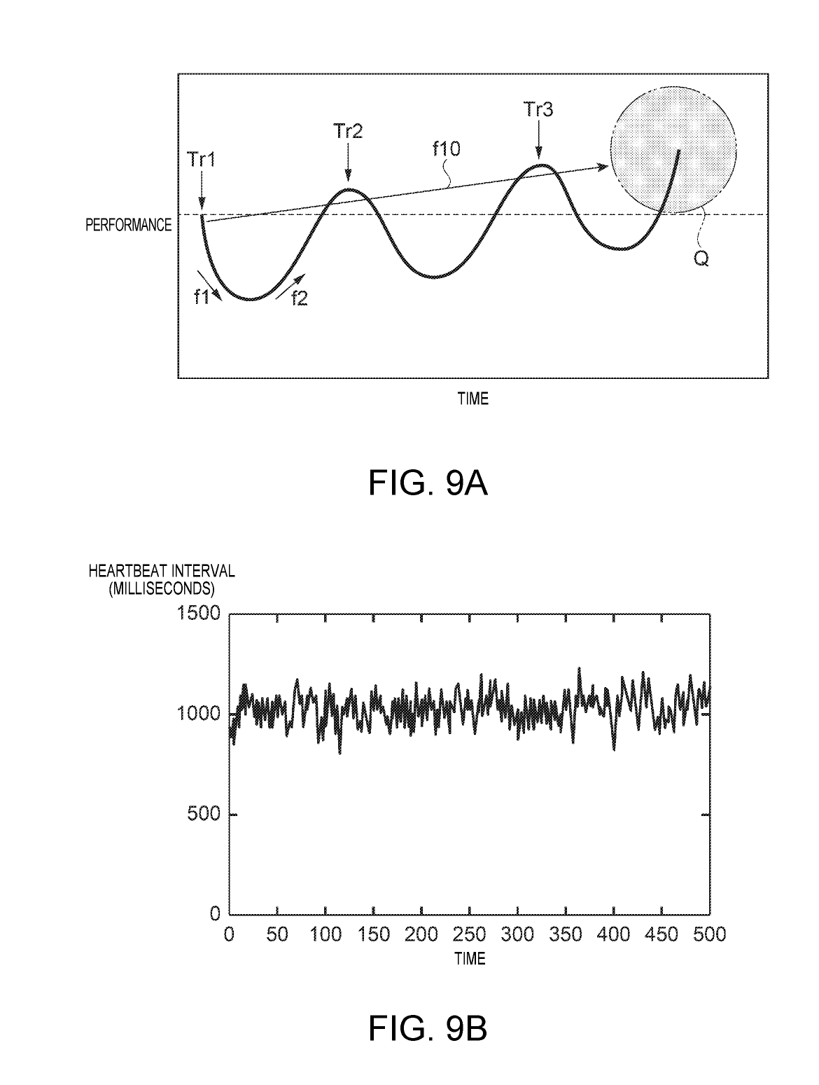

Here, the degree of fatigue of the user, and the HRV, which is a kind of pulse wave information as an indicator indicating the degree of fatigue, will be described referring to FIGS. 6, 7, 8A, 8B, 9A, and 9B. FIG. 6 shows the process of recovery from fatigue (degree of fatigue) of the user, and the correlation between fatigue (degree of fatigue) and time elapsed from training. FIG. 7 explains HRV (heart rate variability). FIG. 8A shows the correlation between performance and time elapsed at the time of performance drop. FIG. 8B is a graph showing HRV (heart rate variability) in the state of a zone P in FIG. 8A. FIG. 9A shows the correlation between performance and time elapsed at the time of performance rise. FIG. 9B is a graph showing HRV (heat rate variability) in the state of a zone Q in FIG. 9A.

First, referring to FIG. 6, fatigue (degree of fatigue) from which recovery is made with the lapse of time after training will be briefly described. FIG. 6 shows the degree of fatigue on the vertical axis and the time elapsed from training on the horizontal axis. The degree of fatigue is divided into the three zones of fatigue state, slightly fatigued, and recovery state. In FIG. 6, the first day is set as a training day. It can be seen that the degree of fatigue is in the zone of fatigue state on the training day, subsequently drops with the lapse of time, then drops to the zone of slightly fatigued on the fifth day, reaches the zone of recovery state on the ninth day, and subsequently drops further. If training is carried out during the lapse of time, the degree of fatigue due to that training is added to the degree of fatigue from which recovery is in progress, resulting in a higher degree of fatigue (fatigue state). Then, from this state of high degree of fatigue (fatigue state), recovery is gradually made with the lapse of time, similarly to the above.

Next, “HRV” used as an indicator indicating the degree of fatigue will be described. HRV is an indicator indicating heart rate variability and is also referred to as heart rate variation. As shown in FIG. 7, HRV is an indicator which grasps, as heart rate variability, the magnitude of the difference between the interval between an R-wave and the next R-wave in changes in time series in heart rate, for example, an interval r1 between R1 and R2 and a subsequent interval r2 between R2 and R3, the difference between the interval r2 between R2 and R3 and a subsequent interval r3 between R3 and R4, the difference between the interval r3 between R3 and R4 and a subsequent interval r4 between R4 and R5, and so forth, that is, the magnitude of the difference in RRI (R-R interval) indicating the time interval (intervals r1 to r4) for each heartbeat, and thus enables determination of the degree of fatigue of the user, based on the magnitude of the variability.

FIG. 8A shows the transition of the degree of fatigue (HRV) in the case where training sessions Tr1, Tr2, Tr3 are carried out in order under the circumstance where sufficient recovery from fatigue is not made (the degree of fatigue is high), that is, where performance is dropping. Performance drops when fatigue is accumulated (the degree of fatigue is high). As shown in FIG. 8A, the second training session Tr2 is carried out under the circumstance where recovery (arrow f2) of performance which has dropped (arrow f1) due to the first training session Tr1 is insufficient. Thus, the degree of fatigue rises again and performance drops. Then, as the third training session Tr3 is carried out under this circumstance of insufficient recovery, performance drops again. In this way, FIG. 8A shows the state where performance gradually drops, as schematically shown by an arrow f5. The transition of the heartbeat interval in this state is shown in FIG. 8B. FIG. 8B shows HRV (heart rate variability) in the state of the zone P in FIG. 8A, that is, the state where the variability is low. In this manner, HRV (heart rate variability) is low at the time of performance drop.

In contrast, FIG. 9A shows the transition of the degree of fatigue (HRV) in the case where training sessions Tr1, Tr2, Tr3 are carried out in order under the circumstance where recovery from fatigue is made (the degree of fatigue is low), that is, where performance is rising. Performance rises when fatigue is not accumulated, in other words, when recovery from fatigue is made (the degree of fatigue is low). As shown in FIG. 9A, the second training session Tr2 is carried out under the circumstance where recovery (arrow f2) of performance which has dropped (arrow f1) due to the first training session Tr1 is made. Thus, the degree of fatigue rises again and performance drops. However, the performance drop is small, and as a general trend, performance rises even if the third training session Tr3 is carried out when recovery from that performance drop is made. In this way, FIG. 9A shows the state where performance gradually rises, as schematically shown by an arrow f10. The transition of the heartbeat interval in this state is shown in FIG. 9B. FIG. 9B shows HRV (heart rate variability) in the state of the zone Q in FIG. 9A, that is, the state where the variability is high. Thus, HRV (heart rate variability) is high at the time of performance rise (under the circumstance where the user is in good physical condition, having recovered from fatigue (degree of fatigue)).

Back to FIG. 5, the processing unit 260 is configured to carry out various kinds of signal processing and control processing, for example, using the storage unit 240 as a work area, and can be realized, for example, by a processor such as CPU or by a logic circuit such as ASIC. The processing unit 260 includes the storage unit 240, a location information acquisition unit 250, the physical condition determination unit 270 for determining the physical condition of the user based on pulse wave information, and a notification processing unit 280.

The storage unit 240 stores pulse wave information of the user and event information that are acquired. The storage unit 240 also stores a program which causes a computer to execute the processing of generating an exercise plan generated using practice day information leading up to the event for the user, an exercise menu and the event information, and modifying the exercise menu or the exercise plan, based on the result of determination on the physical condition of the user acquired from the pulse wave information.

The location information acquisition unit 250 can show the location of the user or provide movement information, for example, based on location information acquired via an antenna 252 from high-frequency radio waves including GPS time information and trajectory information of GPS (global positioning system) satellites, not illustrated, or based on direction information acquired by a direction sensor or the like, not illustrated.

The physical condition determination unit 270, for example, determines the physical condition (degree of fatigue) of the user, based on the pulse wave information such as the HRV of the user acquired by the pulse wave information acquisition unit 210, and transmits the result of the determination to the exercise plan generation unit 230 and to the activity menu acquisition unit 225 via the exercise plan generation unit 230. For example, the physical condition determination unit 270 determines that there is a change in the physical condition of the user if the HRV does not satisfy a predetermined condition (threshold). If the HRV does not satisfy the predetermined condition (threshold), which is set in advance, the physical condition determination unit 270 determines a change in the physical condition of the user and transmits a signal for modifying the exercise menu or the exercise plan.

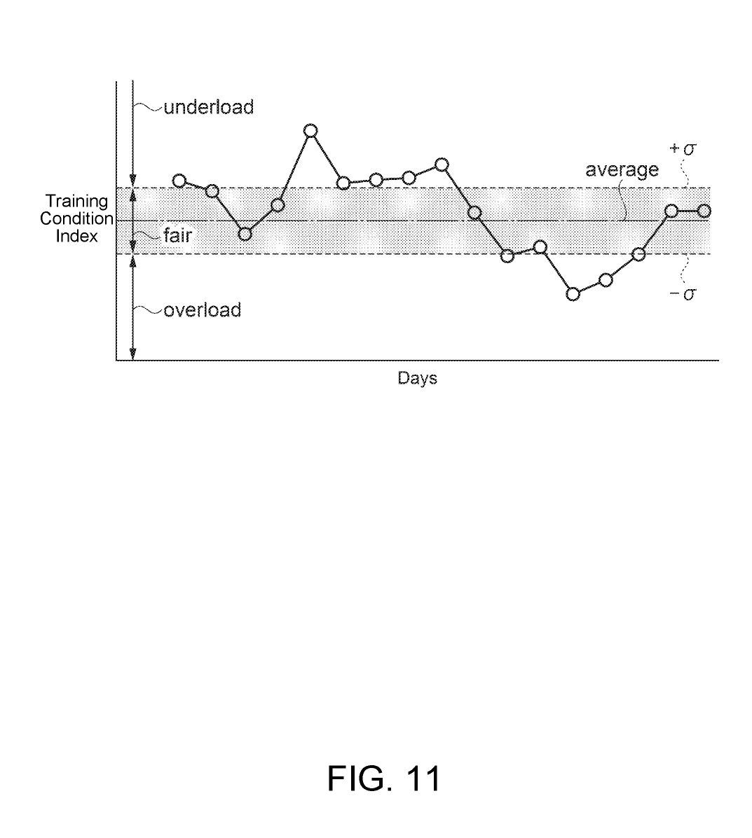

The predetermined condition (threshold) in this case can be that the HRV of the user is within a range including the average value of the HRV (in this example, a deviation value indicating variations of data is used, and standard deviations +σ and −σ from the average value as a point of reference are employed as thresholds), as an example (physical condition determination example 1) shown in the graph of FIG. 11. In this way, a change in the physical condition of the user is determined according to whether the HRV of the user is within a range including the average value of the HRV or not, as the predetermined condition (threshold), in other words, to which side the HRV of the user deviates from this range. Therefore, the constantly changing physical condition of the user can be determined, including variations, and determination can be carried out with a higher degree of certainty. The predetermined condition (threshold) can be set using other techniques, which will be described in detail later in the description of “physical condition determination example 2”.

The notification processing unit 280 carries out control processing to notify the user of the activity menu acquired by the activity menu acquisition unit 225, the exercise plan generated by the exercise plan generation unit 230, and the activity menu and the exercise plan modified based on the result of the determination on the physical condition (degree of fatigue) of the user by the physical condition determination unit 270. The notification processing unit 280 can also carry out control processing to notify the user of the result of the determination on the physical condition (degree of fatigue) of the user by the physical condition determination unit 270. The notification processing unit 280 transmits a notification signal on which control processing is carried out, to the notification unit 290 or to a notification unit 180 provided in another notification device via the communication unit 295.

The notification unit 290 notifies the user of various kinds of information under the control of the notification processing unit 280. The notification unit 290 has a display unit 291 which displays an image and made up of, for example, a liquid crystal display. The notification unit 290 causes the display unit 291 to display an image of the activity menu and the exercise plan, or the modified activity menu and exercise plan, for example, based on the signal from the notification processing unit 280. The notification unit 290 can also have a light emitting unit for notification or a vibration motor (vibrator), as another notification method. In the case of the light emitting unit for notification, the user is notified of various kinds of information by switching on or flashing the light emitting unit. In the case of the vibration motor (vibrator), the user is notified of various kinds of information by the magnitude or duration of vibration. Such information may be provided by the display of an image alone or in combination with at least one of the emission of light for notification and the vibration.

The communication unit 295 carries out communication processing with the notification unit 180 provided in another terminal device or the like, in order to transmit the notification signal on which control processing is carried out by the notification processing unit 280. The communication unit 295 carries out, for example, processing of wireless communication in conformity with a standard such as Bluetooth (trademark registered) or wired communication. The notification signal transmitted in this case can be an image signal, a vibration signal, or a light emission signal or the like.

With the mobile terminal device 300 as an exercise support device, an exercise plan generated by the exercise plan generation unit 230 using the practice day information leading up to the event for the user, the exercise menu, and the event information, can be modified based on the result of determination on the physical condition of the user obtained from pulse wave information by the physical condition determination unit 270. Thus, the user can obtain an exercise menu or an exercise plan which is modified based on the result of the determination on his/her own physical condition obtained from the pulse wave information, and can carry out effective training until the event, preventing overtraining and injuries or the like.

With the mobile terminal device 300, the user can obtain, in advance, detailed information about various conditions such as the number of days until the event, the content of competition, and the venue, elevation above sea level and weather included in the environment information. The user can obtain an exercise menu or an exercise plan based on the detailed information.

With the mobile terminal device 300, the HRV of the user is used as the pulse wave information, and if this HRV does not satisfy a predetermined condition (threshold), a change in the physical condition of the user is determined and the exercise menu or the exercise plan is modified accordingly. Therefore, the user can carry out effective practice (training) until the event, preventing overtraining and injuries or the like.

With the exercise support system 100, the pulse wave information of the user detected by the wearable device 200 as a detection device is processed by the mobile terminal device 300 as an exercise support device, and an exercise menu or an exercise plan which is modified based on the result of determination on the physical condition of the user is provided to the user by the notification unit 180, 290. Thus, the user can obtain the exercise menu or the exercise plan which is modified based on the result of the determination on his/her own physical condition obtained from the pulse wave information, and can carry out effective training until the event, preventing overtraining and injuries or the like.

### 4. Exercise Support Method

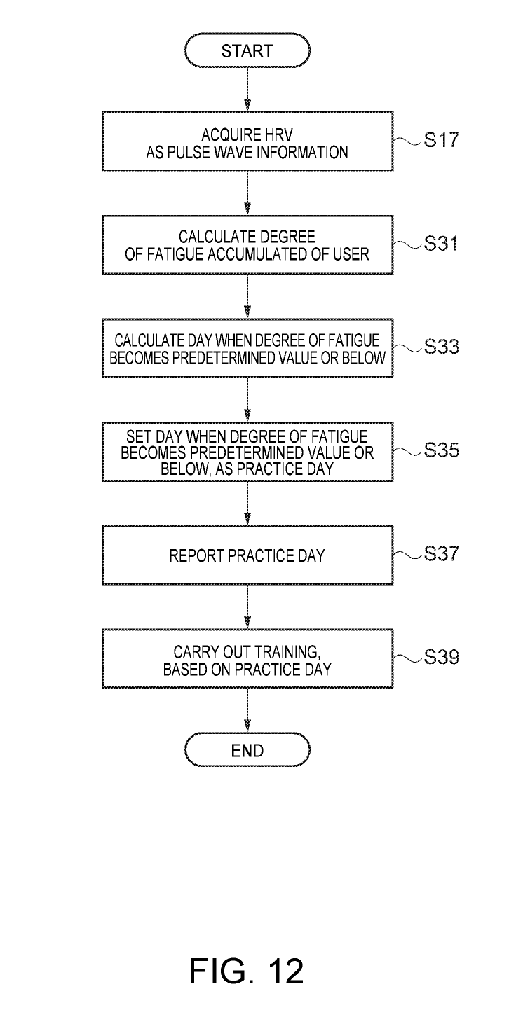

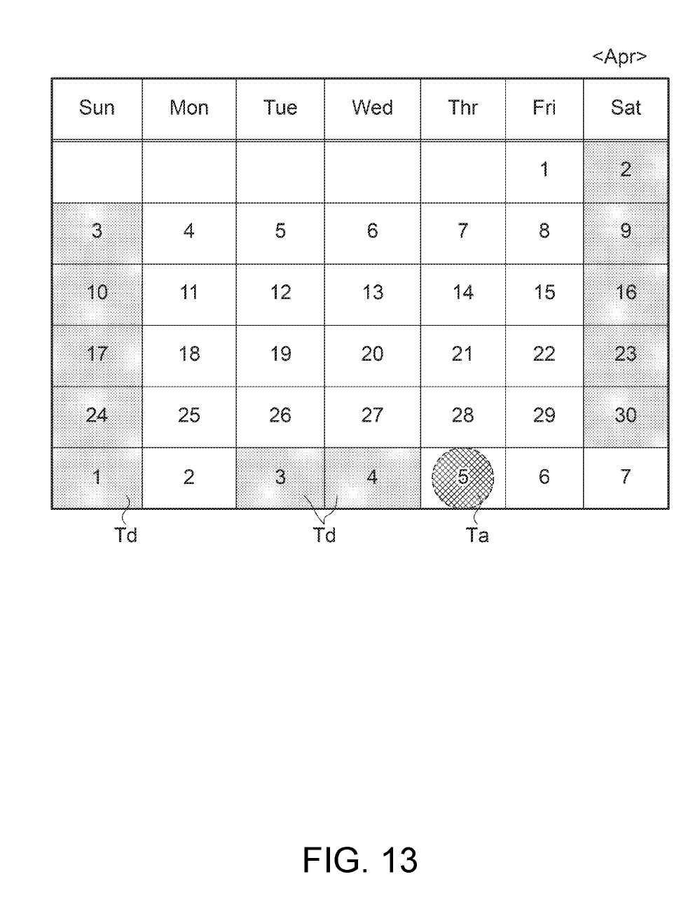

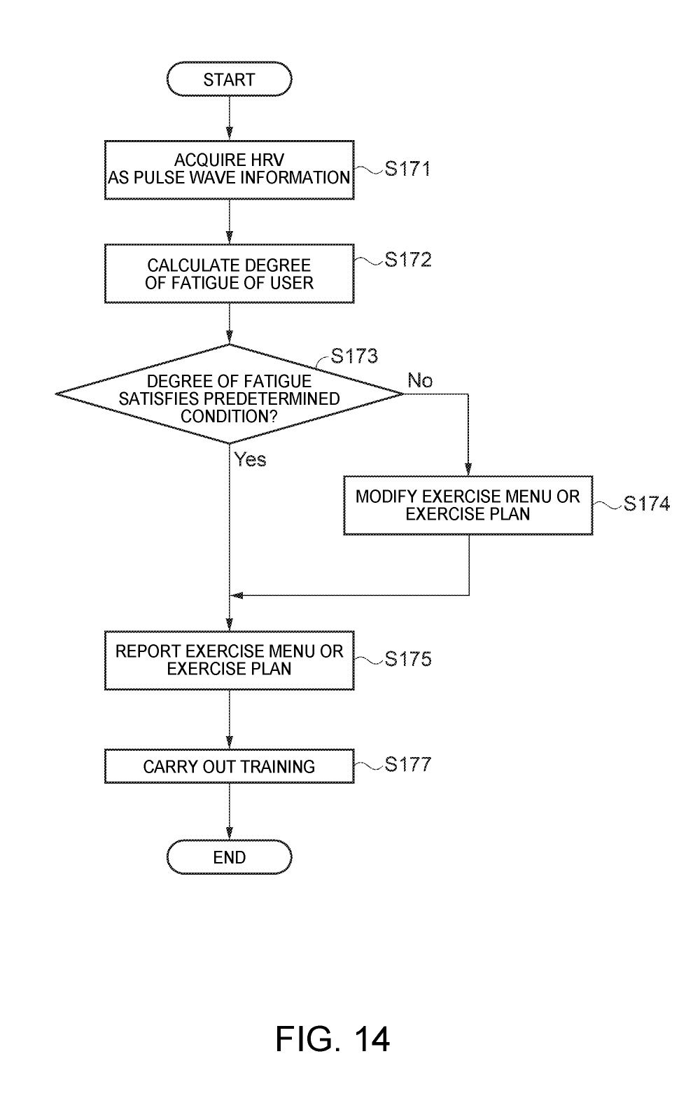

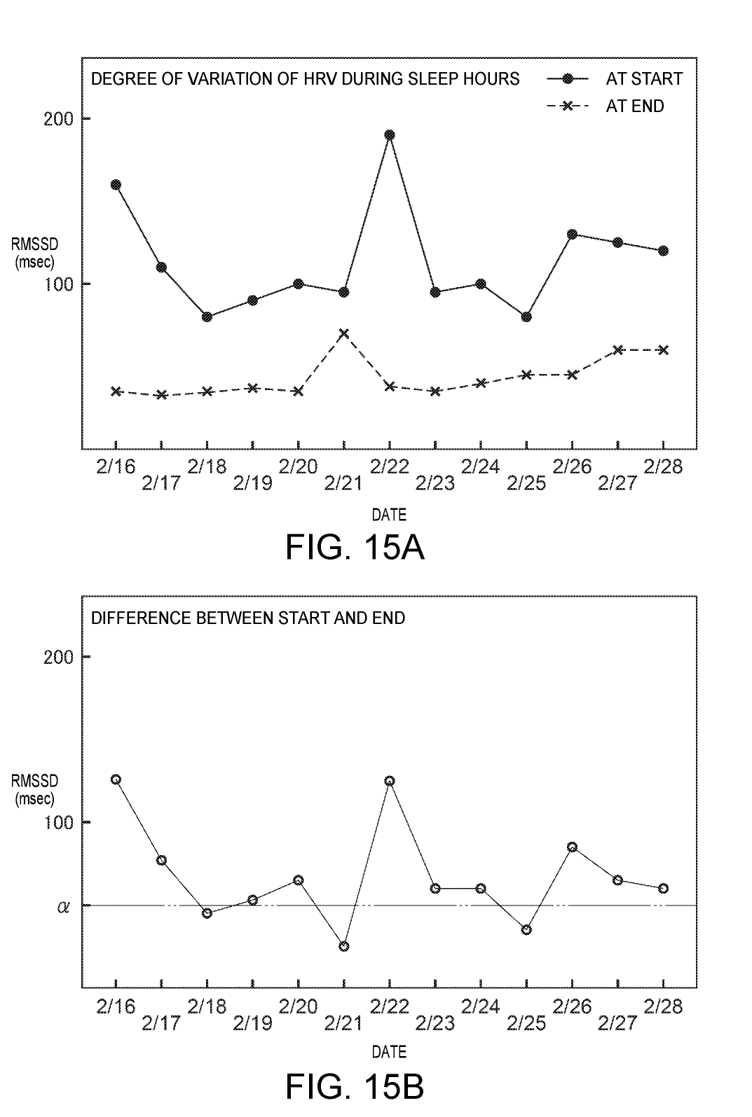

Next, Example 1, Example 2, and Example 3 of an exercise support method will be described, referring to FIGS. 10 to 15B. FIG. 10 is a flowchart showing Example 1 of the exercise support method. FIG. 11 is a graph showing physical condition determination example 1 based on HRV (heart rate variability). FIG. 12 is a flowchart showing Example 2 of the exercise support method. FIG. 13 shows an example of setting practice days. FIG. 14 is a flowchart showing Example 3 of the exercise support method. FIGS. 15A and 15B are graphs for explaining physical condition determination example 2 based on HRV (heart rate variability). FIG. 15A is a first graph. FIG. 15B is a second graph.

### 4.1. Example 1 of Exercise Support Method

Example 1 of the exercise support method includes at least: Step S11 of acquiring event information about an event for the user; Step S13 of acquiring practice day information leading up to the event and an exercise menu; Step S15 of generating an exercise plan using the event information, the practice day information, and the exercise menu; Step S17 of acquiring HRV as pulse wave information of the user; Step S19 and Step S21 of determining the physical condition of the user based on the pulse wave information (HRV); and Step S22 of modifying the exercise menu or the exercise plan, based on the result of the determination in Step S21 of determining the physical condition of the user, as shown in FIG. 10. The order of the respective steps is not limited to that described below and can be rearranged.

Hereinafter, each step of the procedure will be described referring to FIG. 10. With the procedure below, for example, the exercise support method in the case where a user aiming to participate in a competition, for example, a marathon race, uses the exercise support system 100 (wearable device 200 and mobile terminal device 300) in order to carry out effective training until the competition, will be described. In the description of the procedure below, the same reference numbers as those used in the configurations of the wearable device 200 and the mobile terminal device 300 forming the exercise support system 100 are employed.

First, the event information acquisition unit 220 of the mobile terminal device 300 acquires event information about a competition (marathon race) which is an event for the user (Step S11). The acquisition of the event information can be carried out by the user inputting the event information from the input unit 160. The event information includes, for example, at least one of the time and date of the competition (marathon race) in which the user is going to participate, the athletic event (in this example, distance information of the marathon or the like), and environment information (location and elevation above sea level of the venue, ups and downs, climate information of the venue or the like).

Next, the activity menu acquisition unit 225 of the mobile terminal device 300 acquires practice day information leading up to the event for the user and an exercise menu (Step S13). The practice day information acquired by the activity menu acquisition unit 225 can be acquired by at least one of a method in which the user inputs a set practice day and a method in which a practice day is estimated based on past performance information of the user stored in the storage unit 240. Also, schedule information of the user may be acquired and a practice day may be set based on the schedule information.

The exercise menu including the exercise time and the exercise intensity acquired by the activity menu acquisition unit 225 can be decided based on at least one of the physical condition of the user and the environment where the exercise is carried out. The activity menu acquisition unit 225 can also suggest an exercise belonging to the same category as the athletic event included in the acquired event information, as the exercise menu.

Next, the exercise plan generation unit 230 of the mobile terminal device 300 generates an exercise plan, using the acquired event information about the event for the user, the practice day information, and the exercise menu suggested by the activity menu acquisition unit 225 (Step S15). The exercise plan generation unit 230 transmits the generated exercise plan to the notification processing unit 280. The notification processing unit 280 processes the exercise plan transmitted thereto, and the display unit 291 displays an image of this exercise plan as a suggested exercise plan.

Next, the pulse wave information acquisition unit 210 of the mobile terminal device 300 acquires HRV as pulse wave information of the user (Step S17). The acquired HRV is transmitted to the physical condition determination unit 270 as pulse wave information processed by the signal processing unit 215. HRV is an indicator indicating heart rate variability and also referred to as heart rate variation. HRV can indicate the degree of fatigue of the user. HRV is described in detail above and therefore will not be described further in detail here.

Next, the physical condition determination unit 270 determines the physical condition (degree of fatigue) of the user, using the HRV as the pulse wave information of the user transmitted thereto (Step S19). In Step S19 of determining the physical condition (degree of fatigue) of the user, the physical condition determination unit 270 determines whether the HRV satisfies a predetermined condition (threshold) or not (Step S21). If the HRV satisfies the predetermined condition (threshold) (Yes in Step S21), the procedure is followed as it is and the exercise menu or the exercise plan that is suggested in advance is displayed (reported) on the display unit 291 (Step S23). Meanwhile, if the HRV does not satisfy the predetermined condition (threshold) (No in Step S21), it is determined that there is a change in the physical condition of the user, and the exercise menu or the exercise plan is modified (Step S22). Then, the exercise menu or the exercise plan modified in Step S22 of modifying the exercise menu or the exercise plan is displayed (reported) on the display unit 291 (Step S23).

An example of the predetermined condition (threshold) used in Step S21 of determining whether the HRV satisfies the predetermined condition (threshold) or not will be described as determination example 1, referring to FIG. 11. FIG. 11 is a graph showing physical condition determination example 1 based on HRV (heart rate variability). In FIG. 11, the vertical axis represents the value of HRV as the degree of fatigue (training condition) of the user, and the horizontal axis represents the day of measuring HRV.

The predetermined condition (threshold) is that the HRV of the user is within a range of, for example, one standard deviation higher and lower than the average value of the HRV, as shown in the graph of FIG. 11. In this determination example 1, a standard deviation value indicating variation of data is used and the standard deviation +σ and the standard deviation −σ from the average value as a point of reference are used as thresholds. HRV (heart rate variability) is high when the user has recovered from fatigue and is in good physical condition, and low when the user has not recovered from fatigue.

Therefore, if the HRV is above +σ(underload zone shown in FIG. 11), it is determined that the user has recovered from fatigue (low degree of fatigue) and in good physical condition, and therefore can continue carrying out the previously set exercise menu or exercise plan or can set an enhanced exercise menu or exercise plan. In contrast, if the HRV is below −σ (overload zone shown in FIG. 11), it is determined that the user has not recovered from fatigue (high degree of fatigue) and needs to reduce the previously set exercise menu or exercise plan and modify the exercise menu or exercise plan to make the training lighter.

Next, the user carries out the training according to the exercise menu or the exercise plan displayed on the display unit 291 based on his/her own physical condition and the physical condition (degree of fatigue) determined using HRV in Step S19 (Step S24).

With such an exercise support method, an exercise plan is generated, based on practice day information leading up to an event for the user that is acquired, an exercise menu, and event information or the like. Then, the physical condition of the user is determined based on pulse wave information of the user that is acquired. The exercise menu or the exercise plan is modified based on the result of the determination. Thus, the user can obtain the exercise menu or the exercise plan modified based on the result of the determination on his/her own physical condition acquired from the pulse wave information, and can carry out effective training until the event, preventing overtraining and injuries or the like.

The HRV of the user is used as the pulse wave information, and if this HRV does not satisfy a predetermined condition (threshold), a change in the physical condition of the user is determined and the exercise menu or the exercise plan is modified accordingly. Therefore, the user can carry out effective practice (training) until the event, preventing overtraining and injuries or the like.

### 4.2. Example 2 of Exercise Support Method

Example 2 of the exercise support method includes at least: Step S17 of acquiring HRV as pulse wave information of the user; Step S31 of calculating the degree of fatigue of the user based on the pulse wave information (HRV); Step S33 of calculating a day when the degree of fatigue becomes a predetermined value (threshold) or below; Step S35 of setting the calculated day when the degree of fatigue becomes the predetermined value (threshold) or below, as a practice day; and Step S37 of notifying the user of the set practice day, as shown in FIG. 12. Similarly to Example 1, the method in Example 2 also includes: Step S11 of acquiring event information about an event for the user; Step S13 of acquiring practice day information leading up to the event and an exercise menu; and Step S15 of generating an exercise plan, using the event information, the practice day information, and the exercise menu, as described in Example 1, as the steps prior to Step S17 of acquiring HRV as pulse wave information of the user. The description of these steps is omitted. The order of the respective steps is not limited to that described below and can be rearranged.

Hereinafter, each step of the procedure will be described referring to FIG. 12. In the description of the procedure below, the same reference numbers as those used in the configurations of the wearable device 200 and the mobile terminal device 300 forming the exercise support system 100 are employed.

The pulse wave information acquisition unit 210 of the mobile terminal device 300 acquires HRV as pulse wave information of the user (Step S17). The acquired HRV is transmitted to the physical condition determination unit 270 as pulse wave information processed by the signal processing unit 215. HRV is an indicator indicating heart rate variability and also referred to as heart rate variation. HRV can indicate the degree of fatigue of the user. HRV is described in detail above and therefore will not be described further in detail here.

Next, the physical condition determination unit 270 calculates the degree of fatigue accumulated of the user, using the HRV as the pulse wave information of the user transmitted thereto (Step S31). As the user carries out training, the degree of fatigue rises and reaches the fatigue state. Subsequently, recovery is gradually made (the degree of fatigue gradually drops) with the lapse of time. If training is carried out again while the degree of fatigue is dropping, the degree of fatigue due to that training is added to the degree of fatigue from which recovery is being made, and therefore fatigue is accumulated, resulting in a higher degree of fatigue (fatigue state).

Next, the exercise plan generation unit 230 calculates a day when the degree of fatigue becomes a predetermined value (preset threshold) or below, based on the degree of fatigue accumulated of the user calculated by the physical condition determination unit 270 (Step S33).

Next, the exercise plan generation unit 230 sets the day when the degree of fatigue becomes the predetermined value (preset threshold) or below, as a practice day (Step S35). Then, the exercise plan generation unit 230 causes the display unit 291 to display (report) the set practice day (Step S37). FIG. 13 shows a display example of practice days on the display unit 291. In this example, practice days (Td) are indicated by hatching, leading up to the 5^{th} of next month (Ta), which is the day of the competition.

Next, the user carries out training according to the set exercise menu or exercise plan, for example, on the practice days presented as shown in FIG. 13 (Step S39).

With such an exercise support method, the degree of fatigue accumulated in the case where an exercise menu is carried out is calculated based on pulse wave information (HRV), and a day when the degree of fatigue of the user becomes a predetermined value or below is set as a practice day. Therefore, the user can carry out practice (training) on the day when the degree of fatigue is low, that is, on the day when the user is in good physical condition. Thus, the user can carry out effective practice (training) until the event, preventing overtraining and injuries or the like.

### 4.3. Example 3 of Exercise Support Method

Example 3 of the exercise support method includes at least: Step S171 of acquiring HRV as pulse wave information of the user; Step S172 of calculating the degree of fatigue of the user based on the pulse wave information (HRV); Step S173 of determining whether the degree of fatigue satisfies a predetermined value (threshold) or not; Step S174 of modifying the exercise menu or the exercise plan, based on the result of the determination in Step S173; and Step S175 of displaying (reporting) the exercise menu or the exercise plan, as shown in FIG. 14. Similarly to Example 1, the method in Example 3 also includes: Step S11 of acquiring event information about an event for the user; Step S13 of acquiring practice day information leading up to the event and an exercise menu; and Step S15 of generating an exercise plan, using the event information, the practice day information, and the exercise menu, as described in Example 1, as the steps prior to Step S171 of acquiring HRV as pulse wave information of the user. The description of these steps is omitted. The order of the respective steps is not limited to that described below and can be rearranged.

Hereinafter, each step of the procedure will be described referring to FIG. 14. In the description of the procedure below, the same reference numbers as those used in the configurations of the wearable device 200 and the mobile terminal device 300 forming the exercise support system 100 are employed.

The pulse wave information acquisition unit 210 of the mobile terminal device 300 acquires HRV as pulse wave information of the user (Step S171). The acquisition of the HRV is carried out when the user starts sleeping and when the user ends sleeping, as shown in FIG. 15A. FIG. 15A shows a first indicator indicating the degree of variation of the HRV measured when the user starts sleeping and a second indicator indicating the degree of variation of the HRV measured when the user ends sleeping, as the physical condition determination example 2 based on HRV (heart rate variability). The HRV measured at the start of sleep (first indicator) and the HRV measured at the end of sleep (second indicator) are transmitted to the physical condition determination unit 270 as pulse wave information processed by the signal processing unit 215. HRV is an indicator indicating heart rate variability and also referred to as heart rate variation. HRV can indicate the degree of fatigue of the user. HRV is described in detail above and therefore will not be described further in detail here.

Next, the physical condition determination unit 270 calculates the degree of fatigue of the user, using the HRV of the user at the start of sleep (first indicator) and the HRV of the user at the end of sleep (second indicator), transmitted thereto (Step S172). The physical condition determination unit 270 then determines whether the degree of fatigue satisfies a predetermined condition or not (Step S173). In the physical condition determination example 2 based on HRV (heart rate variability), the degree of fatigue of the user or the degree of recovery from fatigue of the user is evaluated, based on the difference between the HRV of the user at the start of sleep (first indicator) and the HRV of the user at the end of sleep (second indicator).

FIG. 15B shows the difference between the HRV of the user measured at the start of sleep (first indicator) and the HRV of the user measured at the end of sleep (second indicator).

Here, if the difference between the HRV at the start of sleep and the HRV at the end of sleep exceeds a threshold a (greater than the threshold a), as shown in FIG. 15B, it is determined that the degree of fatigue of the user satisfies the predetermined condition, that the degree of fatigue is low (recovery is made), and that the user is in good physical condition and can continue carrying out the previously set exercise menu or exercise plan or can set an enhanced exercise menu or exercise plan.

Meanwhile, if the difference between the HRV at the start of sleep and the HRV at the end of sleep does not exceed the threshold a (smaller than the threshold a), it is determined that the degree of fatigue does not satisfy the predetermined condition, that the degree of fatigue is high (recovery is not made), and that the previously set exercise menu or exercise plan needs to be reduced and modified to make the training lighter.

If it is determined in Step S173 that the degree of fatigue satisfies the predetermined condition (Yes in Step S173), the procedure is followed as it is and the previously suggested exercise menu or exercise plan is displayed (reported) on the display unit 291 (Step S175). Meanwhile, if it is determined that the degree of fatigue does not satisfy the predetermined condition (No in Step S173), it is determined that the degree of fatigue of the user or the degree of recovery from fatigue of the user is not good, that is, that the user is not in good physical condition, and therefore the exercise menu or the exercise plan is modified (Step S174).

The exercise menu or the exercise plan modified in Step S174 of modifying the exercise plan is displayed (reported) on the display unit 291 (Step S175).

Next, the user carries out the training according to the exercise menu or the exercise plan displayed on the display unit 291 (Step S177).

With such an exercise support method, the degree of fatigue of the user or the degree of recovery from fatigue is evaluated, based on the difference between the first indicator and the second indicator indicating the degrees of variation of the HRV of the user measured at the start and end of sleep. The user carries out practice (training) according to an exercise plan that is set to be, for example, vigorous (hard) if the degree of recovery from fatigue is sufficient, or light (soft) if the degree of fatigue is high (recovery from fatigue (degree of fatigue) is insufficient), based on the result of the evaluation. Therefore, the user can carry out effective practice (training) until the event, preventing overtraining and injuries or the like. Also, if the degree of fatigue is high (recovery from fatigue (degree of fatigue) is insufficient), the user can rest from training.

What is claimed is:

1. An exercise support device comprising:
a memory storing a program; and
a processor, which when performing the program functions as
an event information acquisition unit which acquires event information about an event in which a user plans to participate;
an activity menu acquisition unit which acquires practice day information identifying the practice day on which the user will exercise before the event, and exercise information representing a value of a parameter of the exercise to be performed on the practice day;
a pulse information acquisition unit which acquires pulse information generated by a pulse sensor about a pulse of the user;
a physical condition determination unit which determines a physical condition of the user based on the pulse information;
an exercise plan generation unit which generates an exercise plan identifying the practice day and the value of the parameter of the exercise to be performed on the practice day, using a result of the determination on the physical condition of the user by the physical condition determination unit, the event information, the practice day information, and the exercise information; and
an output unit which outputs the generated exercise plan to a notification device that notifies the user of the exercise plan,
wherein the exercise plan generation unit modifies the exercise information or the exercise plan, based on the result of the determination by the physical condition determination unit.

2. The exercise support device according to claim 1, wherein the event information includes at least one of a time and a date of the event, an identification of a type of competition to which the event belongs, and environment information.

3. The exercise support device according to claim 1, wherein the activity menu acquisition unit acquires the practice day information by at least one of an input from the user and an estimation operation based on stored past performance information of the user.

4. The exercise support device according to claim 1, wherein
the activity menu acquisition unit determines a value of each of a plurality of parameters of the exercise to be performed including an exercise time and an exercise intensity, based on at least one of the physical condition of the user and an environment where the exercise is performed, and
the activity menu acquisition unit includes the determined values of the parameters in the exercise information.

5. The exercise support device according to claim 1, wherein
the event is a physical competition,
the acquired event information identifies the physical competition as the event, and
the activity menu acquisition unit
determines a suggested exercise to be performed on the practice day belonging to the same category of exercise used in the physical competition indicated in the event information, and
includes the suggested exercise in the exercise information.

6. The exercise support device according to claim 1, wherein the exercise plan generation unit determines the practice day by
calculating a degree of fatigue accumulated by the user in the case where the exercise to be performed is performed, using the pulse information, and
setting a day when the degree of fatigue becomes a predetermined value or below the predetermined value, as the practice day.

7. The exercise support device according to claim 1, wherein
the pulse information is pulse rate variation information of the user,
the physical condition determination unit determines a change in the physical condition of the user if the pulse rate variation information does not satisfy a predetermined condition, and
the exercise plan generation unit modifies the exercise information or the exercise plan, based on a result of the determination by the physical condition determination unit.

8. The exercise support device according to claim 7, wherein the predetermined condition is that the pulse rate variation information of the user is within a range between a standard deviation above an average value of the pulse rate variation information and a standard deviation below the average value of the pulse rate variation information.

9. The exercise support device according to claim 7, wherein
the pulse information includes a first indicator which indicates a degree of variation of the pulse rate variation information measured when the user starts sleeping and a second indicator which indicates a degree of variation of the pulse rate variation information measured when the user ends sleeping,
the physical condition determination unit evaluates the degree of fatigue of the user or a degree of recovery from fatigue of the user, based on a difference between the first indicator and the second indicator, and
the exercise plan generation unit sets the exercise plan, based on a result of the evaluation on the degree of recovery from fatigue.

10. An exercise support method performed by a processor in accordance with a program stored in a memory, comprising:
acquiring event information about an event in which a user plans to participate;
acquiring practice day information identifying the practice day on which the user will exercise before the event, and exercise information representing a value of a parameter of the exercise to be performed on the practice day;
generating an exercise plan identifying the practice day and the value of the parameter of the exercise to be performed on the practice day, using the event information, the practice day information, and the exercise information;
acquiring pulse information generated by a pulse sensor about a pulse of the user;
determining a physical condition of the user based on the pulse information;
modifying the exercise information or the exercise plan, based on a result of the determination in the determining step of the physical condition of the user; and
outputting the generated or modified exercise plan to a notification device that notifies the user of the generated or modified exercise plan.

11. The exercise support method according to claim 10, wherein the event information includes at least one of a time and a date of the event, an identification of a type of competition to which the event belongs, and environment information.

12. The exercise support method according to claim 10, wherein the practice day information is acquired by at least one of an input from the user and an estimation operation based on stored past performance information of the user.

13. The exercise support method according to claim 10, wherein the method further comprises
determining a value of each of a plurality of parameters of the exercise to be performed including an exercise time and an exercise intensity, based on at least one of the physical condition of the user and an environment where the exercise is performed, and
including the determined value of each of the plurality of parameters in the exercise information.

14. The exercise support method according to claim 10, wherein the event is a physical competition and the acquired event information identifies the physical competition to which the event belongs, wherein the method further comprises
determining a suggested exercise to be performed on the practice day belonging to the same category of exercise used in the physical competition identified in the event information, and
including the suggested exercise in the exercise information.

15. The exercise support method according to claim 10, further comprising the step of determining the practice day by
calculating degree of fatigue accumulated by the user in the case where the exercise to be performed is performed, using the pulse information, and
setting a day when the degree of fatigue becomes a predetermined value or below the predetermined value as the practice day.

16. The exercise support method according to claim 10, wherein
the pulse information is pulse rate variation information of the user,
in the determining of the physical condition of the user in the determining step, a change in the physical condition of the user is determined if the pulse rate variation information does not satisfy a predetermined condition, and
in the modifying step for modifying the exercise plan, the exercise information or the exercise plan is modified, based on a result of the determination in the determining step.

17. An exercise support system comprising:
a memory storing a program;
a processor, which when performing the program functions as
a pulse information acquisition unit which acquires pulse information generated by a pulse sensor about a pulse of the user;
a physical condition determination unit which determines a physical condition of the user based on the pulse information;
an event information acquisition unit which acquires event information about an event in which the user plans to participate;
an activity menu acquisition unit which acquires practice day information identifying the practice day on which the user will exercise before the event and exercise information representing a value of a parameter of the exercise to be performed on the practice day;
an exercise plan generation unit which generates an exercise plan identifying the practice day and the value of the parameter of the exercise to be performed on the practice day, using a result of the determination on the physical condition of the user by the physical condition determination unit, the event information, the practice day information, and the exercise information; and a notification device which notifies the user of at least one of the exercise information and the exercise plan.

18. The exercise support system according to claim 17, wherein the exercise plan generation unit modifies the exercise information or the exercise plan, based on a result of the determination by the physical condition determination unit.

19. The exercise support system according to claim 17, wherein the exercise plan generation unit determines the practice day by
calculating a degree of fatigue accumulated by the user in the case where the exercise to be performed is performed, using the pulse information, and
setting a day when the degree of fatigue becomes a predetermined value or below the predetermined value, as the practice day.

20. The exercise support system according to claim 17, wherein
the pulse information is pulse rate variation information of the user,
the physical condition determination unit determines a change in the physical condition of the user if the pulse rate variation information does not satisfy a predetermined condition, and
the exercise plan generation unit modifies the exercise information or the exercise plan, based on a result of the determination by the physical condition determination unit.

21. An exercise support device comprising:
a memory storing a program; and
a processor, which when performing the program functions as
an exercise plan generation unit which generates an exercise plan identifying
i) a practice day on which a user will perform an exercise before an event in which the user will participate, and
ii) a value of a parameter of the exercise to be performed on the practice day,
the exercise plan generation unit identifying the practice day by
identifying the day on which the event will occur from acquired event information,
acquiring exercise information representing a value of a parameter of the exercise to be performed,
calculating the user's accumulated degree of fatigue in the event the user performs the exercise to be performed using pulse information about the user's pulse generated by a pulse sensor, and
selecting a day before the event in which the user's accumulated fatigue becomes no greater than a predetermined value; and
an output unit that outputs the generated exercise plan to a notification device that notifies the user of the exercise plan.

22. The exercise support device according to claim 21,
wherein the exercise plan generation unit modifies the exercise information or the exercise plan, based on the result of the determination by the physical condition determination unit, and
wherein the output unit outputs the modified exercise information or the modified exercise plan.

23. An exercise support device comprising:
a memory storing a program; and
a processor, which when performing the program functions as
an exercise plan generation unit which generates an exercise plan identifying the practice day on which a user will exercise before an event in which the user participates and a value of a parameter of an exercise to be performed on the practice day included in exercise information received by the exercise plan generation unit;
a pulse information acquiring unit which acquires pulse information generated by a pulse sensor about a pulse of the user; and
a physical condition determination unit which determines a physical condition of the user based on the pulse information,
the exercise plan generation unit modifying the exercise information or the exercise plan, based on a result of the determination by the physical condition determination unit of the physical condition of the user; and
an outputting unit which outputs the generated or modified exercise plan to a notification device that notifies the user of the generated or modified exercise plan.
